(19) United States
(12) Patent Application Publication
(10) Pub. No.: US 2026/0038642 A1
(43) Pub. Date: Feb. 5, 2026

(54) SYSTEMS FOR CELLULAR EXPERIMENT DESIGN

(71) Applicant: GMD12, LLC, Longboat Key, FL (US)

(72) Inventor: Brian T. Bennett, Sarasota, FL (US)

(21) Appl. No.: 19/286,809

(22) Filed: Jul. 31, 2025

Related U.S. Application Data
(60) Provisional application No. 63/677,508, filed on Jul. 31, 2024.

(51) Int. Cl. G16C 20/10; G06F 16/3329; G06N 3/08

(52) U.S. Cl. CPC G16C 20/10; G06F 16/3329; G06N 3/08

(57) ABSTRACT

A computer-implemented method for the design of immunofluorescence cellular research tests through a smart assistant in a graphical user interface. The methods include receiving a user-defined molecular pathway and generating an interactive pathway map with functional protein data and assay tool access. The assistant retrieves assay function data containing antibody and fluorophore data, including host species, cross-reactivity, and spectral properties. The user selects and the system displays an assay guide with filtered antibody options for selected proteins and a dye selection guide based on wavelength input. The assistant evaluates fluorophore compatibility with the imaging system using predictive modeling. Finally, it generates a tailored molecular experiment protocol, including selected proteins, antibodies, fluorophores, and optimized testing protocols, each with a confidence score derived from machine learning models trained on historical data.

### CROSS REFERENCE TO RELATED APPLICATION

This application claims priority from U.S. App. No. 63/677,508. The disclosures of the above application are incorporated herein by reference.

### BACKGROUND

Immunofluorescence refers to techniques for detecting and localizing biomolecules, such as proteins, through the use of antibodies conjugated to fluorophores. Antibodies can bind to a specific region of a target protein called an epitope. When performing an immunofluorescence cellular test, a user can select one or more target proteins to be studied. One or more antibodies can then be selected which will specifically bind to the target proteins. The antibodies can be conjugated to fluorophores, which are chemical compounds or groups that can be excited and then release energy in the form of emitted light. The user can treat a cell or tissue sample with the antibodies conjugated to fluorophores and the antibodies will bind to the target proteins. The proteins can then be imaged using various imaging methods. Immunofluorescence can be used in standard microscopy techniques but can also enable advanced super-resolution microscopy techniques, which can provide images having resolutions beyond the diffraction limit. Some examples of these techniques include super-resolution confocal microscopy, structured illumination microscopy (“STIM”), deterministic super-resolution microscopy methods, and stochastic super-resolution microscopy methods.

Despite the useful features of immunofluorescence techniques, the design of cellular tests involving immunofluorescence can be very complex. Designing these tests can involve selecting appropriate antibodies to selectively bind to epitopes of target biomolecules. However, various antibodies may have different selectivity and cross-reactivity which can make certain antibodies appropriate in some circumstances but not in other circumstances. Further, some epitopes are recognized by more than one antibody. Therefore, designing a cellular test can involve selecting antibodies with the highest selectivity while avoiding the use of other antibodies that may bind to the same epitope.

Tests can also be designed to utilize primary immunofluorescence and secondary immunofluorescence. Primary, or direct, immunofluorescence involves a single primary antibody conjugated to a fluorophore for detecting the antibody. Secondary, or indirect, immunofluorescence involves the use of a primary antibody to bind to a target biomolecule and a secondary antibody to bind to the primary antibody, where the secondary antibody is conjugated to a fluorophore. This adds an additional layer of complexity in selecting primary and secondary antibodies.

Further, conjugating a fluorophore to an antibody can sometimes alter the ability of the antibody to specifically recognize and bind to an epitope. Thus, the selection of fluorophores and the combination of the fluorophores with the antibodies also adds complexity in testing design criteria. Fluorophore selection can also involve selecting fluorophores that work best with a certain optical system and imaging process, and also ensuring that different fluorophore are spectrally distinct when imaging multiple different target molecules.

The initial step of selecting target proteins to be imaged can also be very complex where the objective is to investigate information about a cellular pathway. Besides these considerations, immunofluorescence tests also include additional steps such as fixation of a sample, permeabilization of cells, blocking, mounting, performing control experiments, and other factors. Because of the complexity of immunofluorescence testing, it can be difficult to design accurate and reliable tests which provide meaningful results.

Traditional methods for molecular experiment design impose a burden on the user to make decisions regarding species compatibility (i.e., selecting antibodies that will recognize and bind to a target protein), cross-reactivity risks (i.e., an antibody binding to unintended targets that creates false signals and misleading results), fluorophore spectral overlap (i.e., the use of fluorophores with similar excitation or emission wavelengths that can cause signal interference and leads to ambiguous or misleading results and can reduce image clarity and accuracy), and protocol steps. Traditional methods are time-consuming, prone to error, and often rely on trial-and-error to achieve reliable results.

The methods described herein address the complexities of immunofluorescence test design by providing intelligent, computer-assisted methods that streamline and automate critical decision-making steps. The methods integrate AI to provide immunofluorescence experiment design in a graphical user interface. In this way a user is able to interact with a smart assistant through a graphical user interface to create and design an experiment based on a molecular pathway or protein of interest.

The methods simplify the selection of target proteins, antibodies, and fluorophores by dynamically generating a pathway map and integrating a reference assay ruleset (also called assay function data) that accounts for antibody specificity, cross-reactivity, and fluorophore spectral properties. The system reduces the risk of epitope overlap and incompatible antibody-fluorophore combinations by filtering options based on predictive modeling and historical assay data. It also supports both primary and secondary immunofluorescence workflows, guiding users through optimal configurations, in other words the system offers the best possible combination of experimental components and settings (e.g., antibodies, fluorophores, experimental protocols, imaging and microscope systems, as well as post-processing analysis of fluorescent data acquired, and guidelines for interpretation through an ever-growing and significantly large database set). Optimal configuration maximizes the accuracy, reliability, and reproducibility of a test, which are important in scientific research and validation. Additionally, evaluating fluorophore compatibility with specific optical systems and imaging requirements, the invention ensures spectral distinction (i.e., clear differentiation between multiple fluorescent signals in an image) and imaging accuracy. Ultimately, it generates a tailored assay kit list and protocol recommendations, significantly reducing the trial-and-error burden and improving the reliability and reproducibility of immunofluorescence experiments.

### SUMMARY

A system for cellular experiments includes a computing device the runs a machine-learning software module. The machine-learning software module iteratively trains, using training data, a neural network by performing operations that include inserting training data into an iterative training and testing loop to predict a target variable. The target variable is a probability that a molecular experiment protocol meets experiment objective data. In other words, the system is designed to generate an experimental protocol that meets objectives input by an end user.

The machine-learning software module trains by repeatedly determining, during each iteration of the training and testing loop, a target variable, where each iteration of the training and testing loop has differing weights assigned to one or more nodes of the neural network, each of the differing weights being updated with each iteration of the training and testing loop to reduce error in predicting the target variable and to improve predictive ability of the neural network. The result is the creation of a trained neural network. Once trained, the neural network is deployed for use in the system.

The computing device receive interface display data from a network computing device, such as a server. The interface display data has computer readable instructions for generating a graphical user interface. The graphical user interface has an input field that is configured to receive natural language inputs from a user and that is processes by a large language model. The graphical user interface is rendered on an integrated display device coupled to the computing device. The system receive a prompt input in a natural language format from the user that is entered into the input field. The prompt input includes a molecular target identification specifying the material to be analyzed as part of an experiment. The prompt input also includes one or more experiment objectives that the user hopes to achieve.

The system feeds the prompt input to the trained neural network, and the trained neural network outputs the molecular experiment protocol having the highest probability of meeting the experiment objectives. The system also generate material identifications using the prompt input and the assay function data. The material identifications list the materials to be used in the molecular experiment protocol. The computing device renders the molecular experiment protocol and the material identifications on the graphical user interface. The material identifications include an antibody and a fluorophore used in the experiment, and the molecular experiment protocol includes an imaging device identification and imaging device settings used to create images of the molecular target.

The system can also an imaging device configured with the imaging device settings. The user loads the molecular target, the antibody, and the fluorophore into the imaging device and activates the imaging device to generate microscopy image data of the molecular target. The imaging device can be a variety of types of devices, including devices that perform imaging selected from one or more of western blotting, widefield microscopy, structured illumination microscopy, widefield structured illumination microscopy, confocal laser scanning microscopy, super resolution structured illumination microscopy, or stochastic optical reconstruction microscopy.

The system can be configured to design experiments that utilize a multitude of molecular targets, antibodies, and fluorophores. In one embodiment, the system generates material identifications that list a first primary antibody, a first fluorophore, a second primary antibody, a secondary antibody, and one or more additional fluorophores. The molecular experiment protocol further includes instructions specifying an order for reacting the first primary antibody with the first fluorophore and the second primary antibody or secondary antibody with the additional fluorophores. Those of skill in the art will appreciate that the molecular experiment protocol is not limited to two antibody-fluorophore pairings, and additional antibody-fluorophore pairings can be used.

The molecular experiment protocols can include additional experiment steps, such as instructions for cell culturing, cell fixation, cell blocking, antibody incubation, and cellular washing. The molecular experiment protocol can also specify a plurality of imaging device to be used in the experiment along with imaging device settings for each imaging device. In another aspect of the system, the molecular experiment protocol also includes instructions for utilizing a selected buffer, a reagent, and mounting media.

In yet another aspect of the system, the system can generate recommendations, such as refinements to a hypothesis entered as part of the prompt input. The system is also configured to accept inquiries from users seeking information that characterize the molecular target identification and a molecular pathway. The information can include a natural language description of the molecular target function in the molecular pathway.

The system can design experiment protocols for a wide variety of molecular targets that include one or more of Poly (ADP-ribose) polymerase 1 (PARP1), MRE11, RAD50, Nijmegen breakage syndrome 1 (NBS1), Replication Protein A (RPA), Bloom syndrome protein (BLM), Breast cancer 1 (BRCA1), CtBP Interacting Protein or RB Binding Protein 8 (CtIP), RAD51, RAD52, RAD54, FANCN, BRCA2, DSS1, FANCJ (BRIP1 or BACH1), ataxia-telangiectasia mutated (ATM), Tat-interactive protein, 60 kDa (TIP60), Checkpoint kinase 2 (CHK2), BRCA1-Associated RING Domain protein 1 (BARD1), HERC2, H2AX, MDC1, Histone protein 2A (H2A), RNF168, UBC13, Mms2, RNF8, P53-binding protein 1 (53BP1), deubiquitinase DUB (USP28), P53, GADD45, cyclin-dependent kinase inhibitor 1A (aCDKNIA), CIP1, Ccdc98, BRCC36, BRCC45, SUMO.Ub, Small Ubiquitin-like Modifier (SUMO), RNF4, MDC1, and UBC13.

In one embodiment, the system generates and displays a graphical user interface that includes a pathway input field. The system receive a first input from an end user to the pathway input field where the first input specifies a molecular pathway. The system uses the first input to generate a pathway map for the selected molecular pathway that is rendered on the integrated display device. The pathway map includes, without limitation: (i) a plurality of active functional proteins that are connected within the pathway map; (ii) callouts for reach active functional protein, wherein the callouts comprise a description of the one or more active functional proteins; and (iii) an assay guide tool input function.

Selecting the assay guide tool input function renders an assay guide tool on the integrated display device. The assay guide tool includes primary antibody selection options for three or more active functional proteins, such as protein-protein interactions or multiplex/multiprotein complex interactions. The options are filtered using reference assay function data to identify compatible antibodies. The system also displays a dye selection guide on the integrated display device. The dye selection guide includes a list of fluorophores filtered using the reference assay function data and fluorophore data and based on user input of a selected wavelength.

The user enters inputs that specify one or more selected antibodies, one or more selected fluorophores, and an imaging device to be used for imaging. The system evaluates the compatibility of the selected fluorophore with the imaging device and other selected components using a trained neural network. The system also generate an assay kit list that includes the selected one or more active functional proteins, the selected antibodies, the selected fluorophores, and an experiment protocol. The experiment protocol includes imaging device settings for the selected imaging device and a confidence score determined by a machine learning model trained on historical assay data.

In one embodiment, the molecular pathway is one or more of DNA damage and repair, cancer growth and spread, cell cycle, cell death, and mitochondria. The molecular pathway can be a non-homologous end joining double strand break pathway or a homologous recombination double strand break pathway. The dye selection guide displays a list of available dyes at the user wavelength selection and that comply with the reference assay function data.

The system output can take the form of molecular experiment protocols or material identifications that include a first secondary antibody selection, a primary antibody selection, a second secondary antibody selection, a second primary antibody selection, and a third secondary antibody selection. In another aspect of the system, the system output includes a second fluorophore and a third fluorophore where the second fluorophore is bound to the primary antibody selection and the third fluorophore is bound to the second or third secondary antibody selection.

### BRIEF DESCRIPTION OF THE DRAWINGS

Features, aspects, and advantages of the present invention are better understood when the following detailed description of the invention is read with reference to the accompanying figures, in which:

FIG. 1 illustrates an enterprise system, and environment thereof for, according to one or more embodiments of the present invention;

FIG. 2A is a diagram of a feedforward network, according to one or more embodiments, utilized in machine learning;

FIG. 2B is a diagram of a convolution neural network, according to one or more embodiments, utilized in machine learning;

FIG. 2C is a diagram of a portion of the convolution neural network of FIG. 2B, according to one or more embodiments, illustrating assigned weights at connections or neurons;

FIG. 3 is a diagram representing an exemplary weighted sum computation in a node in an artificial neural network;

FIG. 4 is a diagram of a Recurrent Neural Network RNN, according to one or more embodiments, utilized in machine learning;

FIG. 5 is a schematic logic diagram of an artificial intelligence program including a front-end and a back-end algorithm;

FIG. 6 is a flow chart representing a method, according to one or more embodiments, of model development and deployment by machine learning;

FIG. 7 is a flowchart illustrating an example computer-implemented method for designing cellular research tests in a graphical user interface according to one example of the present technology;

FIG. 8 illustrates an example graphical user interface according to another example of the present technology;

FIG. 9 illustrates an example pop-up window of a graphical user interface according to one example of the present technology;

FIG. 10 illustrates an example dynamically responsive pathway map according to another example of the present technology;

FIG. 11 illustrates another example of a dynamically responsive pathway map according to an example of the present technology;

FIG. 12 illustrates another example of a dynamically responsive pathway map according to an example of the present technology;

FIG. 13 illustrates an example assay guide tool according to an example of the present technology;

FIG. 14 illustrates an example dye selection guide according to an example of the present technology; and

FIG. 15 illustrates an example system for designing immunofluorescence cellular research tests in a graphical user interface according to an example of the present technology.

FIG. 16 is an example embodiment of a user's prospective and use of the smart research design assistant (“Identifyn AI”).

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration and not limitation to describe the features and characteristics of the present invention, to set forth the best mode of operation of the invention, and to sufficiently enable one skilled in the art to practice the invention.

Unless described or implied as exclusive alternatives, features throughout the drawings and descriptions should be taken as cumulative, such that features expressly associated with some particular embodiments can be combined with other embodiments.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the herein described embodiments can be configured without departing from the scope and spirit of the invention. Therefore, it is to be understood that, within the scope of the included claims, the invention may be practiced other than as specifically described herein.

Aspects of the present invention are described herein with reference to flowchart illustrations and/or block diagrams of computer-implemented methods and computing systems according to embodiments of the invention. Each block of the flowchart illustrations and/or block diagrams, and combinations of blocks in the flowchart illustrations and/or block diagrams, can be implemented by computer readable program instructions that may be provided to a processor of a general purpose computer, special purpose computer, or other programmable data processing apparatus (the term “apparatus” includes systems and computer program products). The processor may execute the computer readable program instructions thereby creating a means for implementing the actions specified in the flowchart illustrations and/or block diagrams. These computer readable program instructions may also be stored in a computer readable storage medium that can direct a computer and/or other devices to function in a particular manner.

In the flowchart illustrations and/or block diagrams disclosed herein, each block in the flowchart/diagrams may represent a module, segment, or portion of instructions, which comprises one or more executable instructions for implementing the specified logical function(s). In some implementations, the functions noted in the blocks may occur out of the order noted in the Figures. For example, two blocks shown in succession may, in fact, be executed substantially concurrently, or the blocks may sometimes be executed in the reverse order, depending upon the functionality involved.

An application program may be deployed by providing computer infrastructure operable to perform one or more embodiments disclosed herein by integrating computer readable code into a computing system thereby performing the computer-implemented methods disclosed herein. Although various computing environments are described above, these are only examples that can be used to incorporate and use one or more embodiments. Many variations are possible.

It will be understood that relative terms are intended to encompass different orientations or sequences in addition to the orientations and sequences depicted in the drawings and described herein. Relative terminology, such as “substantially” or “about,” describe the specified devices, materials, transmissions, steps, parameters, or ranges as well as those that do not materially affect the basic and novel characteristics of the claimed inventions as whole (as would be appreciated by one of ordinary skill in the art).

The terms “coupled,” “fixed,” “attached to,” “communicatively coupled to,” “operatively coupled to,” and the like refer to both: (i) direct connecting, coupling, fixing, attaching, communicatively coupling; and (ii) indirect connecting coupling, fixing, attaching, communicatively coupling via one or more intermediate components or features, unless otherwise specified herein. “Communicatively coupled to” and “operatively coupled to” can refer to physically and/or electrically related components.

Any steps recited in any method or process claims may be executed in any order and are not limited to the order presented in the claims. Means-plus-function or step-plus-function limitations will only be employed where for a specific claim limitation all of the following conditions are present in that limitation: (i) “means for” or “step for” is expressly recited; and (ii) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### System Level Description

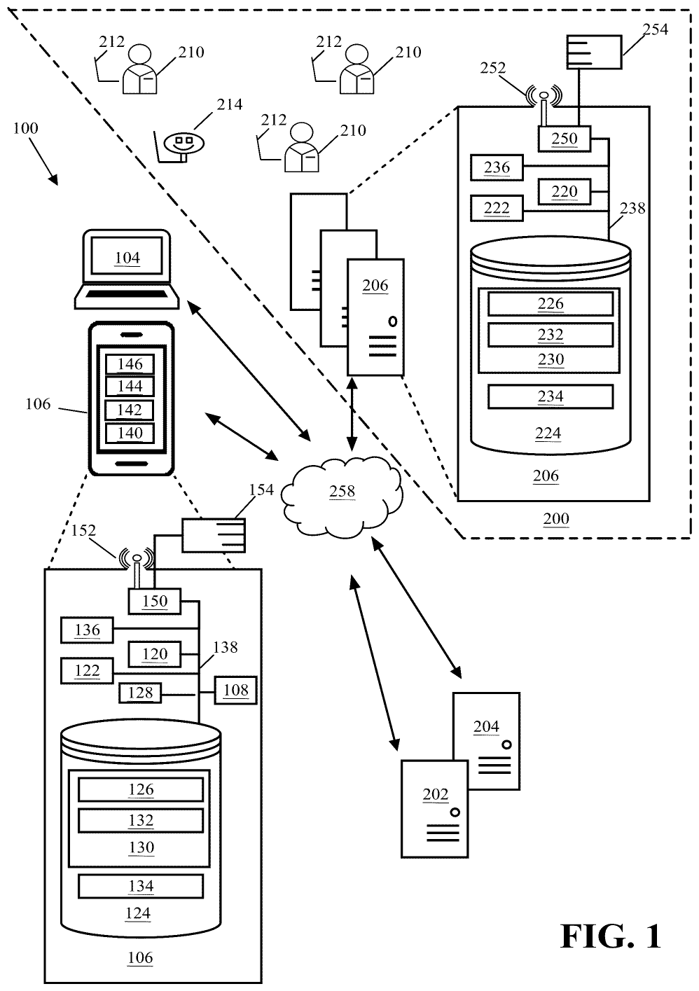

FIG. 1 illustrates an example system 100 according to one or more embodiments where a user 110 interfaces with an enterprise system 200. The environment includes, for example, a distributed cloud computing environment (private cloud, public cloud, community cloud, and/or hybrid cloud), an on-premise environment, fog computing environment, and/or an edge computing environment. The user 110 accesses the enterprise system 200 by use of one or more end user computing devices, such as a personal desktop computing device 104 or a mobile device 106, which may include a smart phone, a tablet computing device, or other portable devices with processing and communication capabilities.

Furthermore, the user device, referring to either or both of the computing device 104 and the mobile device 106, may be or include a workstation, a server, or any other suitable device, including a set of servers, a cloud-based application or system, or any other suitable system, adapted to execute, for example any suitable operating system, including Linux, UNIX, Windows, macOS, IOS, Android and any other known operating system used on personal computers, central computing systems, phones, and other devices.

The user 110 can be an individual, a group, or any entity in possession of, or having access to, the user computing device, referring to either or both of the mobile device 104 and computing device 106, which may be personal or public items. Although the user 110 may be singly represented in some drawings, at least in one or more embodiments according to these descriptions the user 110 is one of many such that a market or community of users, consumers, customers, business entities, government entities, clubs, and groups of any size are all within the scope of these descriptions.

The user device, as illustrated with reference to the mobile device 106, includes components such as, at least one of each of a processing device 120, and a memory device 122 for processing use, such as random access memory (“RAM”), and read-only memory (“ROM”). The illustrated mobile device 106 further includes a storage device 124 including at least one of a non-transitory storage medium, such as a microdrive, for long-term, intermediate-term, and short-term storage of computer-readable instructions 126 for execution by the processing device 120. For example, the instructions 126 can include instructions for an operating system and various applications or programs 130, of which the application 132 is represented as a particular example. The storage device 124 can store various other data items 134, which can include, as non-limiting examples, cached data, user files such as those for pictures, audio and/or video recordings, files downloaded or received from other devices, and other data items preferred by the user or required or related to any or all of the applications or programs 130.

The memory device 122 is operatively coupled to the processing device 120. As used herein, memory includes any computer readable medium to store data, code, or other information. The memory device 122 may include volatile memory, such as volatile Random Access Memory (“RAM”) including a cache area for the temporary storage of data. The memory device 122 may also include embedded or removable non-volatile memory. The non-volatile memory can additionally or alternatively include an electrically erasable programmable read-only memory (“EEPROM”), flash memory or the like.

According to one or more embodiments, the memory device 122 and storage device 124 may be combined into a single storage medium. The memory device 122 and storage device 124 can store any of a number of software applications which comprise computer-executable instructions and code executed by the processing device 120 to implement the functions of the mobile device 106 described herein. For example, the memory device 122 may include software applications such as a web browser application or a virtual telephone application. These software applications also typically provide a graphical user interface (“GUI”) on the display 140 that allows the user 110 to communicate with the mobile device 106 or other devices or systems.

The processing device 120, and other processors described herein, generally include circuitry for implementing communication and/or logic functions of the mobile device 106. For example, the processing device 120 may include a digital signal processor, a microprocessor, and various analog to digital converters, digital to analog converters, and/or other support circuits. Control and signal processing functions of the mobile device 106 are allocated between these devices according to their respective capabilities. The processing device 120 thus may also include the functionality to encode and interleave messages and data prior to modulation and transmission. The processing device 120 can additionally include an internal data modem.

The processing device 120 may include functionality to operate one or more software programs, which may be stored in the memory device 122, or in the storage device 124. For example, the processing device 120 may be capable of operating a connectivity program, such as a web browser application. The web browser application may then allow the mobile device 106 to transmit and receive web content, such as, for example, location-based content and/or other web page content, according to a Wireless Application Protocol (“WAP”), Hypertext Transfer Protocol (“HTTP”), and/or the like.

The processing device 120, in various examples, can operatively perform calculations, can process instructions for execution, and can manipulate information. The processing device 120 can execute machine-executable instructions stored in the storage device 124 and/or memory device 122 to thereby perform methods and functions as described or implied herein, for example by one or more corresponding flow charts expressly provided or implied as would be understood by one of ordinary skill in the art to which the subject matters of these descriptions pertain.

The processing device 120 can be or can include, as non-limiting examples, a central processing unit (“CPU”), a microprocessor, a graphics processing unit (“GPU”), a microcontroller, an application-specific integrated circuit (“ASIC”), a programmable logic device (“PLD”), a digital signal processor (“DSP”), a field programmable gate array (“FPGA”), a state machine, a controller, gated or transistor logic, discrete physical hardware components, and combinations thereof. In one or more embodiments, particular portions or steps of methods and functions described herein are performed in whole or in part by way of the processing device 120, while in other embodiments methods and functions described herein include cloud-based computing in whole or in part such that the processing device 120 facilitates local operations including, as non-limiting examples, communication, data transfer, and user inputs and outputs such as receiving commands from and providing displays to the user.

The mobile device 106, as illustrated, includes an input and output system 136, referring to, including, or operatively coupled with, one or more user input devices and/or one or more user output devices, which are operatively coupled to the processing device 120. The input and output system 136 may include input/output circuitry that may operatively convert analog signals and other signals into digital data, or may convert digital data to another type of signal. For example, the input/output circuitry may receive and convert physical contact inputs, physical movements, or auditory signals (e.g., which may be used to authenticate a user) to digital data. Once converted, the digital data may be provided to the processing device 120.

The input and output system 136 may also include a display 140 (e.g., a liquid crystal display (“LCD”), light emitting diode (“LED”) display, or the like), which can be, as a non-limiting example, a presence-sensitive input screen (e.g., touch screen or the like) of the mobile device 106, which serves both as an output device, by providing graphical and text indicia and presentations for viewing by one or more user 110, and as an input device, by providing virtual buttons, selectable options, a virtual keyboard, and other indicia that, when touched, control the mobile device 106 by user action.

The user output devices include a speaker 144 or other audio device. The user input devices, which allow the mobile device 106 to receive data and actions such as button manipulations and touches from a user such as the user 110, may include any of a number of devices allowing the mobile device 106 to receive data from a user, such as a keypad, keyboard, touch-screen, touchpad, microphone 142, mouse, joystick, other pointer device, button, soft key, infrared sensor, and/or other input device(s). The input and output system 136 may also include a camera 146, such as a digital camera.

Further non-limiting examples of input devices and/or output devices include, one or more of each, any, and all of a wireless or wired keyboard, a mouse, a touchpad, a button, a switch, a light, an LED, a buzzer, a bell, a printer and/or other user input devices and output devices for use by or communication with the user 110 in accessing, using, and controlling, in whole or in part, the user device, referring to either or both of the computing device 104 and a mobile device 106. Inputs by one or more user 110 can thus be made via voice, text or graphical indicia selections. For example, such inputs in some examples correspond to user-side actions and communications seeking services and products of the enterprise system 200, and at least some outputs in such examples correspond to data representing enterprise-side actions and communications in two-way communications between a user 110 and an enterprise system 200.

The input and output system 136 may also be configured to obtain and process various forms of authentication via an authentication system to obtain authentication information of a user 110. Various authentication systems may include, according to one or more embodiments, a recognition system that detects biometric features or attributes of a user such as, for example fingerprint recognition systems and the like (hand print recognition systems, palm print recognition systems, etc.), iris recognition and the like used to authenticate a user based on features of the user's eyes, facial recognition systems based on facial features of the user, DNA-based authentication, or any other suitable biometric attribute or information associated with a user.

Additionally or alternatively, voice biometric systems may be used to authenticate a user using speech recognition associated with a word, phrase, tone, or other voice-related features of the user. Alternate authentication systems may include one or more systems to identify a user based on a visual or temporal pattern of inputs provided by the user. For instance, the user device may display, for example, selectable options, shapes, inputs, buttons, numeric representations, etc. that must be selected in a pre-determined specified order or according to a specific pattern. Other authentication processes are also contemplated herein including, for example, email authentication, password protected authentication, device verification of saved devices, code-generated authentication, text message authentication, phone call authentication, etc. The user device may enable users to input any number or combination of authentication systems.

The user device, referring to either or both of the computing device 104 and the mobile device 106 may also include a positioning device 108, which can be for example a global positioning system device (“GPS”) configured to be used by a positioning system to determine a location of the computing device 104 or mobile device 106. For example, the positioning system device 108 may include a GPS transceiver. In one or more embodiments, the positioning system device 108 includes an antenna, transmitter, and receiver. For example, in one or more embodiments, triangulation of cellular signals may be used to identify the approximate location of the mobile device 106. The positioning device 108 can include a proximity sensor or transmitter, such as an RFID tag, that can sense or be sensed by devices known to be located proximate a merchant or other location to determine that the consumer mobile device 106 is located proximate these known devices.

In the illustrated example, a system intraconnect 138, connects, for example electrically, the various described, illustrated, and implied components of the mobile device 106. The intraconnect 138, in various non-limiting examples, can include or represent, a system bus, a high-speed interface connecting the processing device 120 to the memory device 122, individual electrical connections among the components, and electrical conductive traces on a motherboard common to some or all of the above-described components of the user device (referring to either or both of the computing device 104 and the mobile device 106). The system intraconnect 138 may operatively couple various components with one another, or in other words, electrically connects those components, either directly or indirectly through intermediate components.

The user device, referring to either or both of the computing device 104 and the mobile device 106, with particular reference to the mobile device 106 for illustration purposes, includes a communication interface 150, by which the mobile device 106 communicates and conducts transactions with other devices and systems. The communication interface 150 may include digital signal processing circuitry and may provide two-way communications and data exchanges, for example wirelessly via wireless communication device 152, and for an additional or alternative example, via wired or docked communication by mechanical electrically conductive connector 154.

Communications may be conducted via various modes or protocols, of which GSM voice calls, SMS, EMS, MMS messaging, TDMA, CDMA, PDC, WCDMA, CDMA2000, and GPRS, are all non-limiting and non-exclusive examples. Thus, communications can be conducted, for example, via the wireless communication device 152, which can be or include a radio-frequency transceiver, a Bluetooth device, Wi-Fi device, a Near-field communication device, and other transceivers. In addition, a Global Positioning System (“GPS”) may be included for navigation and location-related data exchanges, ingoing and/or outgoing. Communications may also or alternatively be conducted via the connector 154 for wired connections such by USB, Ethernet, and other physically connected modes of data transfer.

The processing device 120 is configured to use the communication interface 150 as, for example, a network interface to communicate with one or more other devices on a network. In this regard, the communication interface 150 utilizes the wireless communication device 152 as an antenna operatively coupled to a transmitter and a receiver (together a “transceiver”) included with the communication interface 150. The processing device 120 is configured to provide signals to and receive signals from the transmitter and receiver, respectively. The signals may include signaling information in accordance with the air interface standard of the applicable cellular system of a wireless telephone network.

The mobile device 106 further includes a power source 128, such as a battery, for powering various circuits and other devices that are used to operate the mobile device 106. Embodiments of the mobile device 106 may also include a clock or other timer configured to determine and, in some cases, communicate actual or relative time to the processing device 120 or one or more other devices. The clock may facilitate timestamping transmissions, receptions, and other data for security, authentication, logging, polling, data expiry, and forensic purposes.

System 100 as illustrated diagrammatically represents at least one example of a possible implementation, where alternatives, additions, and modifications are possible for performing some or all of the described methods, operations and functions. Although shown separately, in one or more embodiments, two or more systems, servers, or illustrated components may utilized. In some implementations, the functions of one or more systems, servers, or illustrated components may be provided by a single system or server. In one or more embodiments, the functions of one illustrated system or server may be provided by multiple systems, servers, or computing devices, including those physically located at a central facility, those logically local, and those located as remote with respect to each other.

To provide access to, or information regarding, some or all the services and products of the enterprise system 200, automated assistance may be provided by the enterprise system 200. For example, automated access to user accounts and replies to inquiries may be provided by enterprise-side automated voice, text, and graphical display communications and interactions. In at least some examples, any number of human agents 210, can be employed, utilized, authorized or referred by the enterprise system 200. Such human agents 210 can be, as non-limiting examples, customer support representatives, online customer service assistants available to users 110, advisors, managers, sales team members, and referral agents ready to route user requests and communications to preferred or particular other agents, human or virtual.

Human agents 210 may utilize agent computing devices 212 to serve users in their interactions to communicate and take action. The agent devices 212 can be, as non-limiting examples, computing devices, kiosks, terminals, smart devices such as phones, and devices and tools at customer service counters and windows at POS locations. In at least one example, the diagrammatic representation of the components of the user device 106 in FIG. 1 applies as well to one or both of the computing device 104 and the agent devices 212.

Agent computing devices 212 individually or collectively include input devices and output devices, including, as non-limiting examples, a touch screen, which serves both as an output device by providing graphical and text indicia and presentations for viewing by one or more agent 210, and as an input device by providing virtual buttons, selectable options, a virtual keyboard, and other indicia that, when touched or activated, control or prompt the agent device 212 by action of the attendant agent 210. Further non-limiting examples include, one or more of each, any, and all of a keyboard, a mouse, a touchpad, a joystick, a button, a switch, a light, an LED, a microphone serving as input device for example for voice input by a human agent 210, a speaker serving as an output device, a camera serving as an input device, a buzzer, a bell, a printer and/or other user input devices and output devices for use by or communication with a human agent 210 in accessing, using, and controlling, in whole or in part, the agent device 212.

Inputs by one or more human agents 210 can thus be made via voice, text or graphical indicia selections. For example, some inputs received by an agent device 212 in some examples correspond to, control, or prompt enterprise-side actions and communications offering services and products of the enterprise system 200, information thereof, or access thereto. At least some outputs by an agent device 212 in some examples correspond to, or are prompted by, user-side actions and communications in two-way communications between a user 110 and an enterprise-side human agent 210.

From a user perspective experience, an interaction in some examples within the scope of these descriptions begins with direct or first access to one or more human agents 210 in person, by phone, or online for example via a chat session or website function or feature. In other examples, a user is first assisted by a virtual agent 214 of the enterprise system 200, which may satisfy user requests or prompts by voice, text, or online functions, and may refer users to one or more human agents 210 once preliminary determinations or conditions are made or met.

A computing system 206 of the enterprise system 200 may include components such as, at least one of each of a processing device 220, and a memory device 222 for processing use, such as random access memory (“RAM”), and read-only memory (“ROM”). The illustrated computing system 206 further includes a storage device 224 including at least one non-transitory storage medium, such as a microdrive, for long-term, intermediate-term, and short-term storage of computer-readable instructions 226 for execution by the processing device 220. For example, the instructions 226 can include instructions for an operating system and various applications or programs 230, of which the application 232 is represented as a particular example. The storage device 224 can store various other data 234, which can include, as non-limiting examples, cached data, and files such as those for user accounts, user profiles, account balances, and transaction histories, files downloaded or received from other devices, and other data items preferred by the user or required or related to any or all of the applications or programs 230.

The computing system 206, in the illustrated example, includes an input/output system 236, referring to, including, or operatively coupled with input devices and output devices such as, in a non-limiting example, agent devices 212, which have both input and output capabilities.

In the illustrated example, a system intraconnect 238 electrically connects the various above-described components of the computing system 206. In some cases, the intraconnect 238 operatively couples components to one another, which indicates that the components may be directly or indirectly connected, such as by way of one or more intermediate components. The intraconnect 238, in various non-limiting examples, can include or represent, a system bus, a high-speed interface connecting the processing device 220 to the memory device 222, individual electrical connections among the components, and electrical conductive traces on a motherboard common to some or all of the above-described components of the user device.

The computing system 206, in the illustrated example, includes a communication interface 250, by which the computing system 206 communicates and conducts transactions with other devices and systems. The communication interface 250 may include digital signal processing circuitry and may provide two-way communications and data exchanges, for example wirelessly via wireless device 252, and for an additional or alternative example, via wired or docked communication by mechanical electrically conductive connector 254. Communications may be conducted via various modes or protocols, of which GSM voice calls, SMS, EMS, MMS messaging, TDMA, CDMA, PDC, WCDMA, CDMA2000, and GPRS, are all non-limiting and non-exclusive examples. Thus, communications can be conducted, for example, via the wireless device 252, which can be or include a radio-frequency transceiver, a Bluetooth device, Wi-Fi device, Near-field communication device, and other transceivers. In addition, GPS may be included for navigation and location-related data exchanges, ingoing and/or outgoing. Communications may also or alternatively be conducted via the connector 254 for wired connections such as by USB, Ethernet, and other physically connected modes of data transfer.

The processing device 220, in various examples, can operatively perform calculations, can process instructions for execution, and can manipulate information. The processing device 220 can execute machine-executable instructions stored in the storage device 224 and/or memory device 222 to thereby perform methods and functions as described or implied herein, for example by one or more corresponding flow charts expressly provided or implied as would be understood by one of ordinary skill in the art to which the subjects matters of these descriptions pertain. The processing device 220 can be or can include, as non-limiting examples, a central processing unit (“CPU”), a microprocessor, a graphics processing unit (“GPU”), a microcontroller, an application-specific integrated circuit (“ASIC”), a programmable logic device (“PLD”), a digital signal processor (“DSP”), a field programmable gate array (“FPGA”), a state machine, a controller, gated or transistor logic, discrete physical hardware components, and combinations thereof.

Furthermore, the computing system 206, may be or include a workstation, one or more servers, or other suitable device, cloud-based application or system, or any other suitable system, adapted to execute, for example any suitable operating system, including Linux, UNIX, Windows, macOS, IOS, Android, and any known other operating system used on personal computer, central computing systems, phones, and other devices.

The user devices, referring to either or both of the computing device 104 and mobile device 106, the agent devices 212, and the enterprise computing system 206, which may be one or any number centrally located or distributed, are in communication through one or more networks, referenced as network 258 in FIG. 1.

Network 258 provides wireless or wired communications among the components of the system 100 and the environment thereof, including other devices local or remote to those illustrated, such as additional mobile devices, servers, and other devices communicatively coupled to network 258, including those not illustrated in FIG. 1. The network 258 is singly depicted for illustrative convenience, but may include more than one network without departing from the scope of these descriptions. In one or more embodiments, the network 258 may be or provide one or more cloud-based services or operations. The network 258 may be or include an enterprise or secured network, or may be implemented, at least in part, through one or more connections to the Internet.

A portion of the network 258 may be a virtual private network (“VPN”) or an Intranet. The network 258 can include wired and wireless links, including, as non-limiting examples, 802.11a/b/g/n/ac, 802.20, WiMax, LTE, and/or any other wireless link. The network 258 may include any internal or external network, networks, sub-network, and combinations of such operable to implement communications between various computing components within and beyond the illustrated system 100. The network 258 may communicate, for example, Internet Protocol (“IP”) packets, Frame Relay frames, Asynchronous Transfer Mode (“ATM”) cells, voice, video, data, and other suitable information between network addresses. The network 258 may also include one or more local area networks (“LANs”), radio access networks (“RANs”), metropolitan area networks (“MANs”), wide area networks (“WANs”), all or a portion of the internet and/or any other communication system or systems at one or more locations.

The network 258 may incorporate a cloud platform/data center that support various service models including Platform as a Service (“PaaS”), Infrastructure-as-a-Service (“IaaS”), and Software-as-a-Service (“SaaS”). Such service models may provide, for example, a digital platform accessible to the user device (referring to either or both of the computing device 104 and the mobile device 106). Specifically, SaaS may provide a user with the capability to use applications running on a cloud infrastructure, where the applications are accessible via a thin client interface such as a web browser and the user is not permitted to manage or control the underlying cloud infrastructure (i.e., network, servers, operating systems, storage, or specific application capabilities that are not user-specific). PaaS also do not permit the user to manage or control the underlying cloud infrastructure, but this service may enable a user to deploy user-created or acquired applications onto the cloud infrastructure using programming languages and tools provided by the provider of the application. In contrast, IaaS provides a user the permission to provision processing, storage, networks, and other computing resources as well as run arbitrary software (e.g., operating systems and applications) thereby giving the user control over operating systems, storage, deployed applications, and potentially select networking components (e.g., host firewalls).

The network 258 may also incorporate various cloud-based deployment models including private cloud (i.e., an organization-based cloud managed by either the organization or third parties and hosted on-premises or off premises), public cloud (i.e., cloud-based infrastructure available to the general public that is owned by an organization that sells cloud services), community cloud (i.e., cloud-based infrastructure shared by several organizations and manages by the organizations or third parties and hosted on-premises or off premises), and/or hybrid cloud (i.e., composed of two or more clouds, e.g., private community, and/or public).

Two external systems 202 and 204 are illustrated in FIG. 1 that represent any number and variety of data sources, users, consumers, customers, business entities, client systems, government entities, clubs, and groups of any size are all within the scope of the descriptions. In at least one example, the external systems 202 and 204 represent merchant and/or client systems configured to interact with the user device 106 during transactions and also configured to interact with the enterprise system 200 in back-end transactions.

In certain embodiments, one or more of the systems such as the user device (referring to either or both of the computing device 104 and the mobile device 106), the enterprise system 200, and/or the external systems 202 and 204 are, include, or utilize virtual resources. In some cases, such virtual resources are considered cloud resources or virtual machines. The cloud computing configuration may provide an infrastructure that includes a network of interconnected nodes and provides stateless, low coupling, modularity, and semantic interoperability. Such interconnected nodes may incorporate a computer system that includes one or more processors, a memory, and a bus that couples various system components (e.g., the memory) to the processor. Such virtual resources may be available for shared use among multiple distinct resource consumers and in certain implementations, virtual resources do not necessarily correspond to one or more specific pieces of hardware, but rather to a collection of pieces of hardware operatively coupled within a cloud computing configuration so that the resources may be shared as needed.

### Artificial Intelligence Technology

As used herein, an artificial intelligence system, artificial intelligence algorithm, artificial intelligence module, program, and the like, generally refer to computer-implemented programs that are suitable to simulate intelligent behavior (i.e., intelligent human behavior) and/or computer systems and associated programs suitable to perform tasks that typically require a human to perform, such as tasks requiring visual perception, speech recognition, decision-making, translation, and the like. An artificial intelligence system may include, for example, at least one of a series of associated if-then logic statements, a statistical model suitable to map raw sensory data into symbolic categories and the like, or a machine learning program. A machine learning program, machine learning algorithm, or machine learning module, as used herein, is generally a type of artificial intelligence including one or more algorithms that can learn and/or adjust parameters based on input data provided to the algorithm. In some instances, machine learning programs, algorithms, and modules are used at least in part in implementing artificial intelligence (“AI”) functions, systems, and methods.

Artificial Intelligence and/or machine learning programs may be associated with or conducted by one or more processors, memory devices, and/or storage devices of a computing system or device. It should be appreciated that the AI algorithm or program may be incorporated within the existing system architecture or be configured as a standalone modular component, controller, or the like communicatively coupled to the system. An AI program and/or machine learning program may generally be configured to perform methods and functions as described or implied herein, for example by one or more corresponding flow charts expressly provided or implied as would be understood by one of ordinary skill in the art to which the subjects matters of these descriptions pertain.

A machine learning program may be configured to use various analytical tools (e.g., algorithmic applications) to leverage data to make predictions or decisions. Machine learning programs may be configured to implement various algorithmic processes and learning approaches including, for example, decision tree learning, association rule learning, artificial neural networks, recurrent artificial neural networks, long short term memory networks, inductive logic programming, support vector machines, clustering, Bayesian networks, reinforcement learning, representation learning, similarity and metric learning, sparse dictionary learning, genetic algorithms, k-nearest neighbor (“KNN”), and the like. In one or more embodiments, the machine learning algorithm may include one or more image recognition algorithms suitable to determine one or more categories to which an input, such as data communicated from a visual sensor or a file in JPEG, PNG or other format, representing an image or portion thereof, belongs. Additionally or alternatively, the machine learning algorithm may include one or more regression algorithms configured to output a numerical value given an input. Further, the machine learning may include one or more pattern recognition algorithms, e.g., a module, subroutine or the like capable of translating text or string characters and/or a speech recognition module or subroutine. In one or more embodiments, the machine learning module may include a machine learning acceleration logic, e.g., a fixed function matrix multiplication logic, in order to implement the stored processes and/or optimize the machine learning logic training and interface.

Machine learning models are trained using various data inputs and techniques. Example training methods may include, for example, supervised learning, (e.g., decision tree learning, support vector machines, similarity and metric learning, etc.), unsupervised learning, (e.g., association rule learning, clustering, etc.), reinforcement learning, semi-supervised learning, self-supervised learning, multi-instance learning, inductive learning, deductive inference, transductive learning, sparse dictionary learning and the like. Example clustering algorithms used in unsupervised learning may include, for example, k-means clustering, density based special clustering of applications with noise (“DBSCAN”), mean shift clustering, expectation maximization (“EM”) clustering using Gaussian mixture models (“GMM”), agglomerative hierarchical clustering, or the like. According to one or more embodiments, clustering of data may be performed using a cluster model to group data points based on certain similarities using unlabeled data. Example cluster models may include, for example, connectivity models, centroid models, distribution models, density models, group models, graph based models, neural models and the like.

One subfield of machine learning includes neural networks, which take inspiration from biological neural networks. In machine learning, a neural network includes interconnected units that process information by responding to external inputs to find connections and derive meaning from undefined data. A neural network learns to perform tasks by interpreting numerical patterns that take the shape of vectors and by categorizing data based on similarities, without being programmed with any task-specific rules. A neural network generally includes connected units, neurons, or nodes (e.g., connected by synapses) and may allow for the machine learning program to improve performance. A neural network may define a network of functions, which have a graphical relationship. Various neural networks that implement machine learning exist including, for example, feedforward artificial neural networks, perceptron and multilayer perceptron neural networks, radial basis function artificial neural networks, recurrent artificial neural networks, modular neural networks, long short term memory networks, as well as various other neural networks.

Neural networks may perform a supervised learning process where known inputs and known outputs are utilized to categorize, classify, or predict a quality of a future input. However, additional or alternative embodiments of the machine learning program may be trained utilizing unsupervised or semi-supervised training, where none of the outputs or some of the outputs are unknown, respectively. Typically, a machine learning algorithm is trained (e.g., utilizing a training data set) prior to modeling the problem with which the algorithm is associated. Supervised training of the neural network may include choosing a network topology suitable for the problem being modeled by the network and providing a set of training data representative of the problem. Generally, the machine learning algorithm may adjust the weight coefficients until any error in the output data generated by the algorithm is less than a predetermined, acceptable level. For instance, the training process may include comparing the generated output produced by the network in response to the training data with a desired or correct output. An associated error amount may then be determined for the generated output data, such as for each output data point generated in the output layer. The associated error amount may be communicated back through the system as an error signal, where the weight coefficients assigned in the hidden layer are adjusted based on the error signal. For instance, the associated error amount (e.g., a value between −1 and 1) may be used to modify the previous coefficient, e.g., a propagated value. The machine learning algorithm may be considered sufficiently trained when the associated error amount for the output data is less than the predetermined, acceptable level (e.g., each data point within the output layer includes an error amount less than the predetermined, acceptable level). Thus, the parameters determined from the training process can be utilized with new input data to categorize, classify, and/or predict other values based on the new input data.

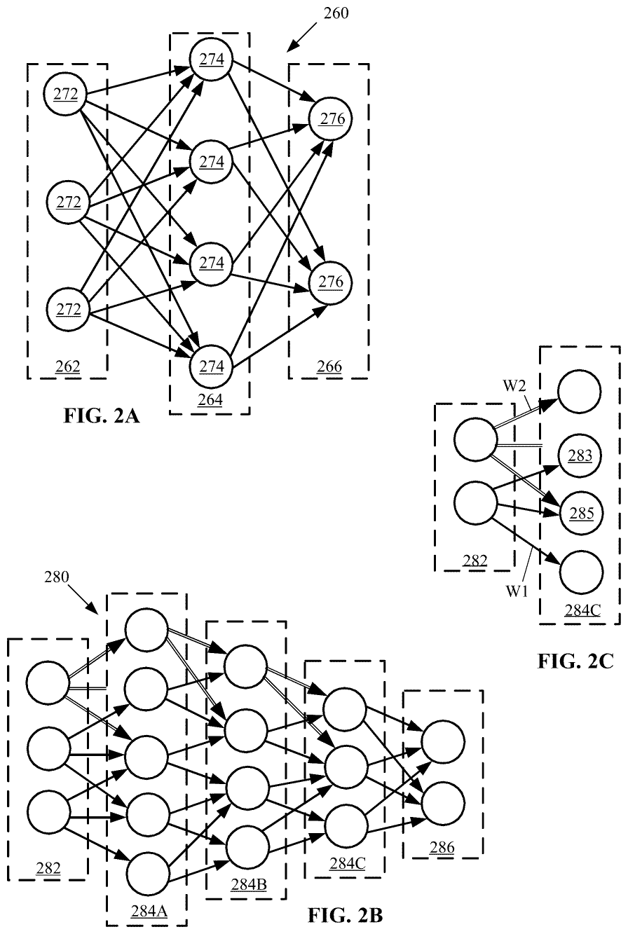

An artificial neural network (“ANN”), also known as a feedforward network, may be utilized, e.g., an acyclic graph with nodes arranged in layers. A feedforward network (see, e.g., feedforward network 260 referenced in FIG. 2A) may include a topography with a hidden layer 264 between an input layer 262 and an output layer 266. The input layer 262, having nodes commonly referenced in FIG. 2A as input nodes 272 for convenience, communicates input data, variables, matrices, or the like to the hidden layer 264, having nodes 274. The hidden layer 264 generates a representation and/or transformation of the input data into a form that is suitable for generating output data. Adjacent layers of the topography are connected at the edges of the nodes of the respective layers, but nodes within a layer typically are not separated by an edge. In one or more embodiments of such a feedforward network, data is communicated to the nodes 272 of the input layer, which then communicates the data to the hidden layer 264. The hidden layer 264 may be configured to determine the state of the nodes in the respective layers and assign weight coefficients or parameters of the nodes based on the edges separating each of the layers, e.g., an activation function implemented between the input data communicated from the input layer 262 and the output data communicated to the nodes 276 of the output layer 266. It should be appreciated that the form of the output from the neural network may generally depend on the type of model represented by the algorithm. Although the feedforward network 260 of FIG. 2A expressly includes a single hidden layer 264, other embodiments of feedforward networks within the scope of the descriptions can include any number of hidden layers. The hidden layers are intermediate the input and output layers and are generally where all or most of the computation is done.

An additional or alternative type of neural network suitable for use in the machine learning program and/or module is a Convolutional Neural Network (“CNN”). A CNN is a type of feedforward neural network that may be utilized to model data associated with input data having a grid-like topology. In one or more embodiments, at least one layer of a CNN may include a sparsely connected layer, in which each output of a first hidden layer does not interact with each input of the next hidden layer. For example, the output of the convolution in the first hidden layer may be an input of the next hidden layer, rather than a respective state of each node of the first layer. CNNs are typically trained for pattern recognition, such as speech processing, language processing, and visual processing. As such, CNNs may be particularly useful for implementing optical and pattern recognition programs required from the machine learning program. A CNN includes an input layer, a hidden layer, and an output layer, typical of feedforward networks, but the nodes of a CNN input layer are generally organized into a set of categories via feature detectors and based on the receptive fields of the sensor, retina, input layer, etc. Each filter may then output data from its respective nodes to corresponding nodes of a subsequent layer of the network. A CNN may be configured to apply the convolution mathematical operation to the respective nodes of each filter and communicate the same to the corresponding node of the next subsequent layer. As an example, the input to the convolution layer may be a multidimensional array of data. The convolution layer, or hidden layer, may be a multidimensional array of parameters determined while training the model.

An exemplary convolutional neural network CNN is depicted and referenced as 280 in FIG. 2B. As in the basic feedforward network 260 of FIG. 2A, the illustrated example of FIG. 2B has an input layer 282 and an output layer 286. However where a single hidden layer 264 is represented in FIG. 2A, multiple consecutive hidden layers 284A, 284B, and 284C are represented in FIG. 2B. The edge neurons represented by white-filled arrows highlight that hidden layer nodes can be connected locally, such that not all nodes of succeeding layers are connected by neurons. FIG. 2C, representing a portion of the convolutional neural network 280 of FIG. 2B, specifically portions of the input layer 282 and the first hidden layer 284A, illustrates that connections can be weighted. In the illustrated example, labels W1 and W2 refer to respective assigned weights for the referenced connections. Two hidden nodes 283 and 285 share the same set of weights W1 and W2 when connecting to two local patches.

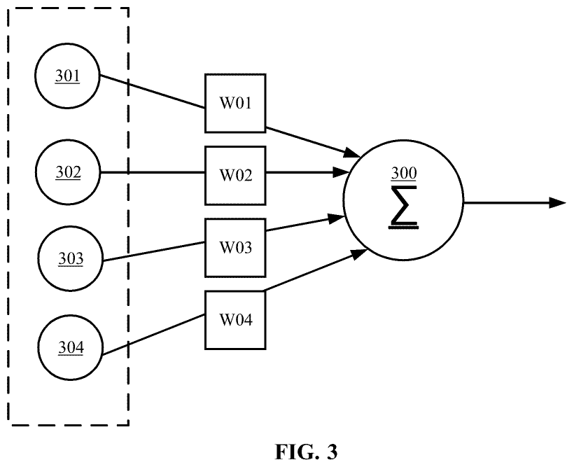

Weight defines the impact a node in any given layer has on computations by a connected node in the next layer. FIG. 3 represents a particular node 300 in a hidden layer. The node 300 is connected to several nodes in the previous layer representing inputs to the node 300. The input nodes 301, 302, 303 and 304 are each assigned a respective weight W01, W02, W03, and W04 in the computation at the node 300, which in this example is a weighted sum.

An additional or alternative type of feedforward neural network suitable for use in the machine learning program and/or module is a Recurrent Neural Network (“RNN”). An RNN may allow for analysis of sequences of inputs rather than only considering the current input data set. RNNs typically include feedback loops/connections between layers of the topography, thus allowing parameter data to be communicated between different parts of the neural network. RNNs typically have an architecture including cycles, where past values of a parameter influence the current calculation of the parameter, e.g., at least a portion of the output data from the RNN may be used as feedback/input in calculating subsequent output data. In one or more embodiments, the machine learning module may include an RNN configured for language processing, e.g., an RNN configured to perform statistical language modeling to predict the next word in a string based on the previous words. The RNN(s) of the machine learning program may include a feedback system suitable to provide the connection(s) between subsequent and previous layers of the network.

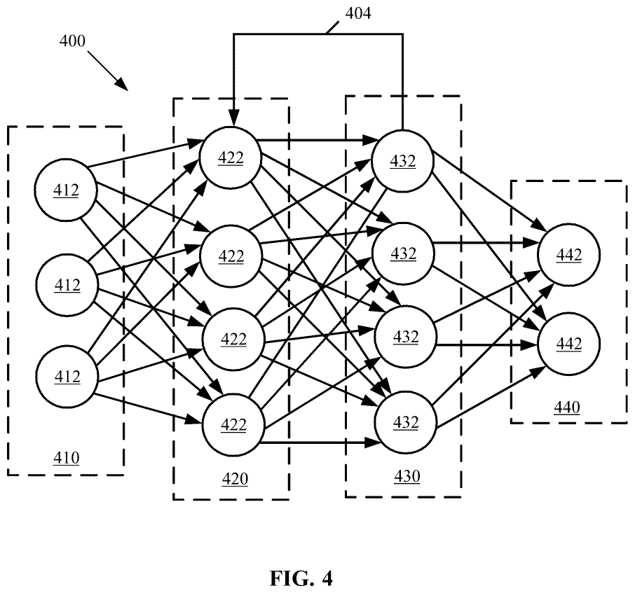

An example for a Recurrent Neural Network RNN is referenced as 400 in FIG. 4. As in the basic feedforward network 260 of FIG. 2A, the illustrated example of FIG. 4 has an input layer 410 (with nodes 412) and an output layer 440 (with nodes 442). However, where a single hidden layer 264 is represented in FIG. 2A, multiple consecutive hidden layers 420 and 430 are represented in FIG. 4 (with nodes 422 and nodes 432, respectively). As shown, the RNN 400 includes a feedback connector 404 configured to communicate parameter data from at least one node 432 from the second hidden layer 430 to at least one node 422 of the first hidden layer 420. It should be appreciated that any or all of the nodes of a subsequent layer may provide or communicate a parameter or other data to a previous layer of the RNN 400. Moreover and in one or more embodiments, the RNN 400 may include multiple feedback connectors 404 (e.g., connectors 404 suitable to communicatively couple pairs of nodes and/or connector systems 404 configured to provide communication between three or more nodes). Additionally or alternatively, the feedback connector 404 may communicatively couple two or more nodes having at least one hidden layer between them, i.e., nodes of nonsequential layers of the RNN 400.

In an additional or alternative embodiment, the machine-learning program may include one or more support vector machines. A support vector machine may be configured to determine a category to which input data belongs. For example, the machine-learning program may be configured to define a margin using a combination of two or more of the input variables and/or data points as support vectors to maximize the determined margin. Such a margin may generally correspond to a distance between the closest vectors that are classified differently. The machine-learning program may be configured to utilize a plurality of support vector machines to perform a single classification. For example, the machine-learning program may determine the category to which input data belongs using a first support vector determined from first and second data points/variables, and the machine-learning program may independently categorize the input data using a second support vector determined from third and fourth data points/variables. The support vector machine(s) may be trained similarly to the training of neural networks, e.g., by providing a known input vector (including values for the input variables) and a known output classification. The support vector machine is trained by selecting the support vectors and/or a portion of the input vectors that maximize the determined margin.

As depicted, and in one or more embodiments, the machine-learning program may include a neural network topography having more than one hidden layer. In such embodiments, one or more of the hidden layers may have a different number of nodes and/or the connections defined between layers. In one or more embodiments, each hidden layer may be configured to perform a different function. As an example, a first layer of the neural network may be configured to reduce a dimensionality of the input data, and a second layer of the neural network may be configured to perform statistical programs on the data communicated from the first layer. In one or more embodiments, each node of the previous layer of the network may be connected to an associated node of the subsequent layer (dense layers). Generally, the neural network(s) of the machine-learning program may include a relatively large number of layers, e.g., three or more layers, and may be referred to as deep neural networks. For example, the node of each hidden layer of a neural network may be associated with an activation function utilized by the machine-learning program to generate an output received by a corresponding node in the subsequent layer. The last hidden layer of the neural network communicates a data set (e.g., the result of data processed within the respective layer) to the output layer. Deep neural networks may require more computational time and power to train, but the additional hidden layers provide multistep pattern recognition capability and/or reduced output error relative to simple or shallow machine learning architectures (e.g., including only one or two hidden layers).

According to various implementations, deep neural networks incorporate neurons, synapses, weights, biases, and functions and can be trained to model complex non-linear relationships. Various deep learning frameworks may include, for example, TensorFlow, MxNet, PyTorch, Keras, Gluon, and the like. Training a deep neural network may include complex input/output transformations and may include, according to one or more embodiments, a backpropagation algorithm. According to one or more embodiments, deep neural networks may be configured to classify images of handwritten digits from a dataset or various other images. According to one or more embodiments, the datasets may include a collection of files that are unstructured and lack predefined data model schema or organization. Unlike structured data, which is usually stored in a relational database (“RDBMS”) and can be mapped into designated fields, unstructured data comes in many formats that can be challenging to process and analyze. Examples of unstructured data may include, according to non-limiting examples, dates, numbers, facts, emails, text files, scientific data, satellite imagery, media files, social media data, text messages, mobile communication data, and the like.

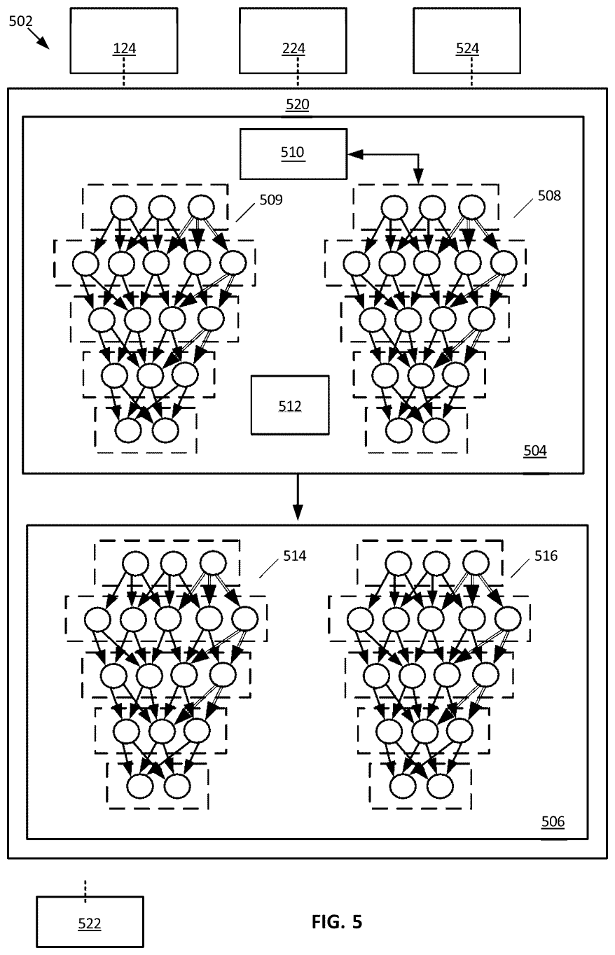

Referring now to FIG. 5 and one or more embodiments, an AI program 502 may include a front-end algorithm 504 and a back-end algorithm 506. The artificial intelligence program 502 may be implemented on an AI processor 520, such as the processing device 120, the processing device 220, and/or a dedicated processing device. The instructions associated with the front-end algorithm 504 and the back-end algorithm 506 may be stored in an associated memory device and/or storage device of the system (e.g., storage device 124, memory device 122, storage device 224, and/or memory device 222) communicatively coupled to the AI processor 520, as shown. Additionally or alternatively, the system may include one or more memory devices and/or storage devices (represented by memory 524 in FIG. 5) for processing use and/or including one or more instructions necessary for operation of the AI program 502. In one or more embodiments, the AI program 502 may include a deep neural network (e.g., a front-end algorithm 504 configured to perform pre-processing, such as feature recognition, and a back-end algorithm 506 configured to perform an operation on the data set communicated directly or indirectly to the back-end algorithm 506 that together form the deep neural network). For instance, the front-end algorithm 504 can include at least one CNN 508 communicatively coupled to send output data to the back-end algorithm 506.

Additionally or alternatively, the front-end algorithm 504 can include one or more AI algorithms 510, 512 (e.g., statistical models or machine learning programs such as decision tree learning, associate rule learning, recurrent artificial neural networks, support vector machines, and the like). In various embodiments, the front-end algorithm 504 may be configured to include built in training and inference logic or suitable software to train the neural network prior to use (e.g., machine learning logic including, but not limited to, image recognition, mapping and localization, autonomous navigation, speech synthesis, document imaging, or language translation such as natural language processing). For example, a CNN 508 and/or AI algorithm 510 may be used for image recognition, input categorization, and/or support vector training. In one or more embodiments and within the front-end algorithm 504, an output from an AI algorithm 510 may be communicated to a CNN 508 or 509, which processes the data before communicating an output from the CNN 508, 509 and/or the front-end algorithm 504 to the back-end algorithm 506. In various embodiments, the back-end algorithm 506 may be configured to implement input and/or model classification, speech recognition, translation, and the like. For instance, the back-end network 506 may include one or more CNNs (e.g., CNN 514) or dense networks (e.g., dense networks 516), as described herein.

For instance and in one or more embodiments of the AI program 502, the program may be configured to perform unsupervised learning, in which the machine learning program performs the training process using unlabeled data, e.g., without known output data with which to compare. During such unsupervised learning, the neural network may be configured to generate groupings of the input data and/or determine how individual input data points are related to the complete input data set (e.g., via the front-end algorithm 504). For example, unsupervised training may be used to configure a neural network to generate a self-organizing map, reduce the dimensionally of the input data set, and/or to perform outlier/anomaly determinations to identify data points in the data set that falls outside the normal pattern of the data. In one or more embodiments, the AI program 502 may be trained using a semi-supervised learning process in which some but not all of the output data is known, e.g., a mix of labeled and unlabeled data having the same distribution.

In one or more embodiments, the AI program 502 may be accelerated via a machine-learning framework 522 (e.g., hardware). The machine learning framework may include an index of basic operations, subroutines, and the like (primitives) typically implemented by AI and/or machine learning algorithms. Thus, the AI program 502 may be configured to utilize the primitives of the framework 522 to perform some or all of the calculations required by the AI program 502. Primitives suitable for inclusion in the machine learning framework 522 include operations associated with training a convolutional neural network (e.g., pools), tensor convolutions, activation functions, basic algebraic subroutines and programs (e.g., matrix operations, vector operations), numerical method subroutines and programs, and the like.

It should be appreciated that the machine-learning program may include variations, adaptations, and alternatives suitable to perform the operations necessary for the system, and the present disclosure is equally applicable to such suitably configured machine learning and/or artificial intelligence programs, modules, etc. For instance, the machine-learning program may include one or more long short-term memory (“LSTM”) RNNs, convolutional deep belief networks, deep belief networks DBNs, and the like. DBNs, for instance, may be utilized to pre-train the weighted characteristics and/or parameters using an unsupervised learning process. Further, the machine-learning module may include one or more other machine learning tools (e.g., Logistic Regression (“LR”), Naive-Bayes, Random Forest (“RF”), matrix factorization, and support vector machines) in addition to, or as an alternative to, one or more neural networks, as described herein.

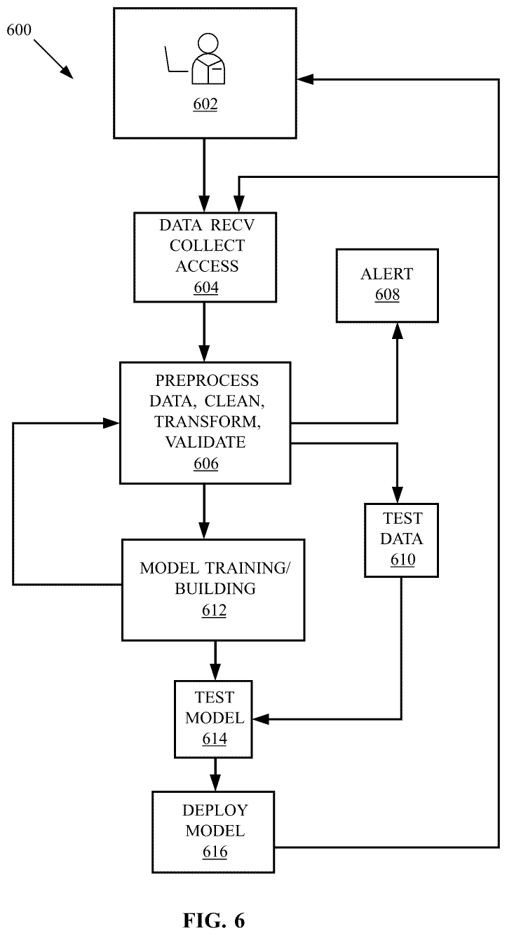

FIG. 6 is a flow chart representing a method 600, according to one or more embodiments, of model development and deployment by machine learning. The method 600 represents at least one example of a machine learning workflow in which steps are implemented in a machine-learning project.

In step 602, a user authorizes, requests, manages, or initiates the machine-learning workflow. This may represent a user such as human agent, or customer, requesting machine-learning assistance or AI functionality to simulate intelligent behavior (such as a virtual agent) or other machine-assisted or computerized tasks that may, for example, entail visual perception, speech recognition, decision-making, translation, forecasting, predictive modelling, and/or suggestions as non-limiting examples. In a first iteration from the user perspective, step 602 can represent a starting point. However, with regard to continuing or improving an ongoing machine learning workflow, step 602 can represent an opportunity for further user input or oversight via a feedback loop. Such feedback may flow through a user, or in various embodiments, the method automatically provides feedback, retrains and redeploys the retrained model.

In step 604, data is received, collected, accessed, or otherwise acquired and entered as can be termed data ingestion. In step 606, the data ingested in step 604 is pre-processed, for example, by cleaning, and/or transformation such as into a format that the following components can digest. The incoming data may be versioned to connect a data snapshot with the particularly resulting trained model. As newly trained models are tied to a set of versioned data, preprocessing steps are tied to the developed model. If new data is subsequently collected and entered, a new model will be generated. If the preprocessing step 606 is updated with newly ingested data, an updated model will be generated. Step 606 can include data validation, which focuses on confirming that the statistics of the ingested data are as expected, such as that data values are within expected numerical ranges, that data sets are within any expected or required categories, and that data comply with any needed distributions such as within those categories. Step 606 can proceed to step 608 to automatically alert the initiating user, other human or virtual agents, and/or other systems, if any anomalies are detected in the data, thereby pausing or terminating the process flow until corrective action is taken.

In step 610, training test data such as a target variable value is inserted into an iterative training and testing loop. In step 612, model training, a core step of the machine learning work flow, is implemented. A model architecture is trained in the iterative training and testing loop. For example, features in the training test data are used to train the model based on weights and iterative calculations in which the target variable may be incorrectly predicted in an early iteration as determined by comparison in step 614, where the model is tested. Subsequent iterations of the model training in step 612 are conducted with updated weights in the calculations.

During each iteration of the training and testing loop, the accuracy of the model may be evaluated. In to one or more embodiments, the re-evaluation of the model can include comparing an output of the model with an actual target result or variable to determine the accuracy of the prediction. If the model is not satisfying a minimum threshold level of accuracy (i.e., the model is underfitted), the system may automatically determine that the threshold level of accuracy is not satisfied and may adjust the weights for a subsequent iteration of the training and testing loop.

The weights may be iteratively adjusted during each iteration of the training and testing loop based on the comparison to the threshold level of accuracy. However, there is a balance for training the model to avoid overfitting when the model would not perform well on predictions of new data. Rather, the model is automatically trained to be well-fitted such that it satisfies a threshold level of accuracy without learning the noise in the data to the extent that the model would not apply to new data by preventing additional iterations of the training and testing once a maximum accuracy threshold value has been obtained. Thus, with each iteration of the training and testing loop, the accuracy of the model is improved and the iterative training and testing of the model provides an improvement to the performance of a computer and computing technology because the system may automatically determine how many iterations to perform so that the model is well-fitted by surpassing the minimum threshold level of accuracy while automatically stopping the iterative training and testing of the model before the maximum accuracy threshold is obtained.

In one or more embodiments, the training and testing loop utilizes a backpropagation algorithm and a gradient descent algorithm. Gradient descent is an optimization algorithm used to minimize differentiable real-valued multivariate functions. Gradient descent is an optimization algorithm used to minimize differentiable real-valued multivariate functions. The gradient descent algorithm may be used to iteratively adjust model parameters using calculated derivatives to minimize a loss function. Backpropagation may be used to calculate the gradient of the error function with respect to the neural network's weights.

When compliance and/or success in the model testing in step 614 is achieved, process flow proceeds to step 616, where model deployment is triggered. The model may be utilized in AI functions and programming, for example to simulate intelligent behavior, to perform machine-assisted or computerized tasks, of which visual perception, speech recognition, decision-making, translation, forecasting, predictive modelling, and/or automated suggestion generation serve as non-limiting examples.

As discussed above, oversight of a deployed machine learning model may be automatically performed via a feedback loop whereby the method assesses performance of the deployed model (see step 616) and the feedback loop automatically provides feedback for further training of the machine learning model to improve its performance, and upon completion of the other method steps such as 612, the machine learning model that has been automatically retrained based on the feedback loop is then redeployed (step 614). In one or more embodiments, the system is continually receiving training data as new predictions are made and more data is collected. The continuous training data may be discretized to generate input data to retrain the model. Discretization methods can convert continuous data to discrete data by binning, clustering, and numerical discretization. The model may monitor incoming data sets to make predictions. When predictions are made, the system analyzes the predictions to determine whether the model needs to be retrained.

In one or more embodiments, the model may detect anomalies in the predictions. Anomaly detection can provide a benefit by identifying instances of the prediction that deviate from expected data or a general pattern. A difficulty in anomaly detection is that the system must define the boundary between ordinary data and anomalous data to accurately classify the data as ordinary or anomalous. The line between ordinary and anomalous may be difficult to determine with cases approaching a boundary and based on the specific application. For example, small variations may trigger an identification of an anomaly in the data while relatively larger deviations may be considered normal in less sensitive applications. The disclosed systems and methods may provide solutions to detecting anomalies in order to more accurately and quickly determine whether a model needs to be retrained. If data would be inapplicable or would corrupt the model by reducing the quality of the input data or training process (e.g., due to missing values, outliers, inconsistent formatting, incorrect labels, noisy data, etc.) that data may be automatically dropped and the source of that data may be blocked from providing data that would be used to train the model. This reflects an improvement in the process of training and deploying a model that is accurate and specific to the type of prediction sought. This provides an improvement in the field of model training, which provides a practical application.

In other applications, the anomaly detections processes described herein may be used to provide enhanced security to the overall computing system by detecting malicious attacks on network security. For example, the system may take proactive measures to remediate danger by detecting the source address associated with potentially malicious packets and dropping potentially malicious packets. This provides an improvement in network security by dropping potentially malicious packets and blocking future traffic from the source address of the potentially malicious source address.

### Natural Language Processing Technology

The systems and methods disclosed herein may also be used to analyze text to form the predictions. In particular, the systems and methods described herein include a combination of elements that are utilized in a specific manner for automatically performing automated processes based on technological efficiency, which provides a specific improvement over prior art systems resulting in improved computer processing for faster automated processing functions. For example, the systems and method may apply robotic process automation for digital transformation of the data based on specific criteria to interpret text and unstructured data using text processing software techniques. The interpretation of the text may be implemented using the models described herein including unsupervised learning techniques or supervised learning techniques. The processor may track how much memory and/or processing time has been allocated to perform a function and the system may be trained to automatically detect and identify processes eligible for increased efficiencies based on existing inefficiencies in the process.

For example, the machine learning models may use unsupervised learning to identify and characterize hidden structures of unstructured and unlabeled content data, or supervised techniques that operate on labeled content data and include instructions informing the system which outputs are related to specific input values. In such instances, software processing can rely on iterative training techniques and training data to configure neural networks to understand individual words, phrases, subjects, sentiments, and parts of speech.

Supervised learning software systems are trained using content data that is labeled or “tagged.” During training, the supervised software systems learn the best mapping function between a known data input and expected known output (i.e., labeled or tagged content data). Supervised natural language processing software uses the best approximating mapping learned during training to analyze unforeseen input data (never seen before) to accurately predict the corresponding output. Supervised learning software systems often require extensive and iterative optimization cycles to adjust the input-output mapping until they converge to an expected and well-accepted level of performance, such as an acceptable threshold error rate between a calculated probability and a desired threshold probability.

The software systems are supervised because the way of learning from training data mimics the same process of a teacher supervising the end-to-end learning process. Supervised learning software systems are typically capable of achieving excellent levels of performance, but this excellent level of performance requires labeled data to be available. Developing, scaling, deploying, and maintaining accurate supervised learning software systems can take significant time, resources, and technical expertise from a team of skilled data scientists. Moreover, precision of the systems is dependent on the availability of labeled content data for training that is comparable to the corpus of content data that the system will process in a production environment.

Supervised learning software systems implement techniques that include, without limitation, Latent Semantic Analysis (“LSA”), Probabilistic Latent Semantic Analysis (“PLSA”), Latent Dirichlet Allocation (“LDA”), and more recent Bidirectional Encoder Representations from Transformers (“BERT”). Latent Semantic Analysis software processing techniques process a corporate of content data files to ascertain statistical co-occurrences of words that appear together, which then give insights into the subjects of those words and documents.

Unsupervised learning software systems can perform training operations on unlabeled data and less requirement for time and expertise from trained data scientists. Unsupervised learning software systems can be designed with integrated intelligence and automation to automatically discover information, structure, and patterns from content data. Unsupervised learning software systems can be implemented with clustering software techniques that include, without limitation, K-means clustering, Mean-Shift clustering, Density-based clustering, Spectral clustering, Principal Component Analysis, and Neural Topic Modeling (“NTM”).

Clustering software techniques can automatically group semantically similar words together to accelerate the derivation and verification of an underneath common intent—i.e., ascertain or derive a new classification or subject, and not just classification into an existing subject or classification. Unsupervised learning software systems are also used for association rules mining to discover relationships between features from content data.

The system can incorporate an interactive content software service that utilizes one or more supervised or unsupervised software processing techniques to perform a subject classification analysis to generate subject data. Suitable software processing techniques can include, without limitation, LSA, PLSA, and LDA. Latent Semantic Analysis software processing techniques generally process a corpus of alphanumeric text files, or documents, to ascertain statistical co-occurrences of words that appear together, which then give insights into the subjects of those words and documents. The interactive content software service can utilize software processing techniques that include Non-Matrix Factorization, Correlated Topic Model (“CTM”), and K-Means or other types of clustering.

Neural networks may be trained using training set content data that comprise sample tokens, phrases, sentences, paragraphs, or documents for which desired subjects, content sources, interrogatories, or sentiment values are known. A labeling analysis may be performed on the training set content data to annotate the data with known subject labels (e.g., topics discussed during a shared experience also referred to herein as topic identifications), interrogatory labels (e.g., questions asked during a shared experience also referred to herein as interrogatory identifications), content source labels (e.g., identifications of participants to a shared experience), segment labels (e.g., opening, issue identification, or another segment), or sentiment labels, thereby generating annotated training set content data. For example, a person can utilize a labeling software application to review training set content data to identify and tag or “annotate” various parts of speech, subjects, questions, content sources, and sentiments.

The training set content data is fed to neural networks to identify subjects, content sources, or sentiments and the corresponding probabilities. For example, the analysis might identify that particular text represents a question with a 35% probability. If the annotations indicate the text is, in fact, a question, the error rate is 65% or the difference between the calculated probability and the known certainty. Then parameters to the neural network are adjusted (i.e., constants and formulas that implement the nodes and connections between node), to increase the probability from 35% to ensure the neural network produces more accurate results, thereby reducing the error rate. The process is run iteratively on different sets of training set content data to increase the accuracy of the neural network.

The content data is first pre-processes using a reduction analysis to create reduced content data. The reduction analysis first performs a qualification operation that removes unqualified content data that does not meaningfully contribute to the subject classification analysis. The qualification operation removes certain content data according to criteria defined by a provider. For instance, the qualification analysis can determine whether content data files are “empty” and contain no recorded linguistic expressions between a provider agent and a user and designate such empty files as not suitable for use in a subject classification analysis. As another example, the qualification analysis can designate files below a certain size or having a shared experience duration below a given threshold (e.g., less than one minute) as also being unsuitable for use in an analysis, such as subject identification, sentiment analysis, or segmentation.

The reduction analysis can also perform a contradiction operation to remove contradictions and punctuations from the content data. Contradictions and punctuation include removing or replacing abbreviated words or phrases that can cause inaccuracies in a subject classification analysis. Examples include removing or replacing the abbreviations “min” for minute, “u” for you, and “wanna” for “want to,” as well as apparent misspellings, such as “mssed” for the word missed. In one or more embodiments, the contradictions can be replaced according to a standard library of known abbreviations, such as replacing the acronym “brb” with the phrase “be right back.” The contradiction operation can also remove or replace contractions, such as replacing “we're” with “we are.”

The reduction analysis can also streamline the content data by performing one or more of the following operations, including: (i) tokenization to transform the content data into a collection of words or key phrases having punctuation and capitalization removed; (ii) stop word removal where short, common words or phrases such as “the” or “is” are removed; (iii) lemmatization where words are transformed into a base form, like changing third person words to first person and changing past tense words to present tense; (iv) stemming to reduce words to a root form, such as changing plural to singular; and (v) hyponymy and hypernym replacement where certain words are replaced with words having a similar meaning so as to reduce the variation of words within the content data.

Following a reduction analysis, the reduced content data is vectorized to map the alphanumeric text into a vector or matrix form—an operation that is also known as embedding. The content data files are converted to a series of machine encoded communication elements that make up the vectors or matrices (referred to herein as vectors as a shorthand even where matrices can be used). Machine encoded communication elements, also referred to as “communication elements” or sometimes “words,” can be words, phrases, symbols (e.g., an emoji, logo, etc.), numbers, or other elements of that make up a written or transcribed communication.

One approach to vectorizing content data includes applying “bag-of-words” modeling. The bag-of-words approach counts the number of times a particular word appears in content data to convert the words into a numerical value. The bag-of-words model can include parameters, such as setting a threshold on the number of times a word must appear to be included in the vectors.

Another technique for vectorization includes the Word2Vec method. The Word2Vec method employs skip-grams or a continuous bag of words (“CBOW”) and reconstructs the linguistic context of words by considering both the order of words in history as well as the predicted order of words. Shallow neural networks that have an input layer, an output layer, and a projection layer, iterate over a corpus of text to learn the association between the machine encoded communication elements. The method assumes neighboring words in a text have semantic similarities with each other, and semantically similar machine encoded communication elements are mapped to geometrically close embedding vectors. Sematic similarity is measured using the cosine similarity metric. Cosine similarity is equal to the cosine of an angle where the angle is measured between the vector representation of text (e.g., sentences, phrases, or documents). If the cosine angle is one, it means that the words are overlapping. If the cosine angle is a right angle, the machine encoded communication elements hold no contextual similarity and are independent of each other.

Techniques to encode the content communication elements may, in part, determine how often communication elements appear together. Communication elements are often individual words but can also be groups of works like phrases, speech patterns, tone, cadence, or other characteristics of text. Determining the adjacent pairing of communication elements can be achieved by creating a co-occurrence matrix with the value of each member of the matrix counting how frequently one communication element coincides with another, either just before or just after it. That is, the words or communication elements form the row and column labels of a matrix, and a numeric value appears in matrix elements that correspond to a row and column label for communication elements that appear adjacent in the content data.

As an alternative to counting communication elements (e.g., words) in a corpus of content data and turning it into a co-occurrence matrix, another software processing technique may be used where a communication element in the content data corpus predicts the next communication element. Looking through a corpus, counts may be generated for adjacent communication elements, and the counts are converted from frequencies into probabilities (i.e., using n-gram predictions with Kneser-Ney smoothing) using a simple neural network. Suitable neural network architectures include a skip-gram architecture. The neural network may be trained by feeding through a large corpus of content data, and embedded middle layers in the neural network are adjusted to best predict the next word.

The predictive processing creates weight matrices that densely carry contextual, and hence semantic, information from the selected corpus of content data. Pre-trained, contextualized content data embedding can have high dimensionality. To reduce the dimensionality, a uniform manifold approximation and projection algorithm (“UMAP”) can be applied to reduce dimensionality while maintaining essential information.

Prior to conducting a subject analysis to ascertain subject identifications in the content data (i.e., topics or subjects addressed in the content data) or interaction driver identifications in the content data (i.e., reasons why the customer initiated the interaction with the provider, such as the reason underlying a support request), the system can perform a concentration analysis on the content data. The concentration analysis concentrates, or increases the density of, the content data by identifying and retaining communication elements that have significant weight in the subject analysis and discarding or ignoring communication elements that have relativity little weight.

In one or more embodiments, the concentration analysis includes executing a term frequency-inverse document frequency (“tf-idf”) software processing technique to determine the frequency or corresponding weight quantifier for communication elements with the content data. The weight quantifiers are compared against a pre-determined weight threshold to generate concentrated content data that is made up of communication elements having weight quantifiers above the weight threshold.

Vectorization can be better understood with reference to the following simplified example. A corpus of machine encoded communication elements might include the following where each sentence is a row in a matrix: [I, forgot, my, account, password∥The, account, is, locked∥Please, reset, my, password, and, account]. Each machine encoded communication element can then be replaced by its frequency, such as: [1, 1, 2, 3, 2∥1, 3, 1, 1∥1, 1, 2, 2, 1, 3]. Here, the highest frequency is three, so each frequency value is divided by 3 to yield: [0.33, 0.33, 0.66, 1, 0.66∥0.33, 1, 0.33, 0.33∥0.33, 0.33, 0.66, 0.66, 0.33, 1].

In other examples, the vectorization creates a “sparse matrix” where each sentence, or row of the matrix, includes a frequency value for all distinct machine encoded communication elements within the corpus of content data. Where a communication element does not appear in a sentence, the frequency of the communication element is set to zero. Continuing with the foregoing example, the distinct communication elements include [I, forgot, my, account, password, the, is, locked, please, reset, and]. Each sentence is represented as follows: [1, 1, 1, 1, 1, 0, 0, 0, 0, 0, 0∥0, 0, 0, 1, 0, 1, 1, 1, 0, 0, 0∥1, 1, 2, 2, 1, 3∥0, 1, 1, 1, 0, 0, 0, 1, 1, 1].

Techniques to encode the context of words, or machine encoded communication elements, determine how often machine encoded communication elements appear together. Determining the adjacent pairing of machine encoded communication elements can be achieved by creating a co-occurrence matrix with the value of each member of the matrix counting how often one machine encoded communication element coincides with another, either just before or just after it. That is, the words or machine encoded communication elements form the row and column labels of a matrix, and a numeric value appears in matrix elements that correspond to a row and column label for communication elements that appear adjacent in the content data.

The concentrated content data is processed using a subject classification analysis to determine subject identifications (i.e., topics) addressed within the content data. The subject classification analysis can specifically identify one or more interaction driver identifications that are the reason why a user initiated a shared experience or support service request. An interaction driver identification can be determined by, for example, first determining the subject identifications having the highest weight quantifiers (e.g., frequencies or probabilities) and comparing such subject identifications against a database of known interaction driver identifications.

In one or more embodiments, the subject classification analysis is performed on the content data using a LDA analysis to identify subject data that includes one or more subject identifications (e.g., topics addressed in the underlying content data). Performing the LDA analysis on the reduced content data may include transforming the content data into an array of text data representing key words or phrases that represent a subject (e.g., a bag-of-words array) and determining the one or more subjects through analysis of the array. Each cell in the array can represent the probability that given text data relates to a subject. A subject is then represented by a specified number of words or phrases having the highest probabilities (i.e., the words with the five highest probabilities), or the subject is represented by text data having probabilities above a predetermined subject probability threshold.

Clustering software processing techniques include K-means clustering, which is an unsupervised processing technique that does not utilized labeled content data. Clusters are defined by “K” number of centroids where each centroid is a point that represents the center of a cluster. The K-means processing technique run in an iterative fashion where each centroid is initially placed randomly in the vector space of the dataset, and the centroid moves to the center of the points that is closest to the centroid. In each new iteration, the distance between each centroid and the points are recalculated, and the centroid moves again to the center of the closest points. The processing completes when the position or the groups no longer change or when the distance in which the centroids change does not surpass a pre-defined threshold.

The clustering analysis yields a group of words or communication elements associated with each cluster, which can be referred to as subject vectors. Subjects may each include one or more subject vectors where each subject vector includes one or more identified communication elements (i.e., keywords, phrases, symbols, etc.) within the content data as well as a frequency of the one or more communication elements within the content data. The interactive content software service can be configured to perform an additional concentration analysis following the clustering analysis that selects a pre-defined number of communication elements from each cluster to generate a descriptor set, such as the five or ten words having the highest weights in terms of frequency of appearance (or in terms of the probability that the words or phrases represent the true subject when neural networking architecture is used). In one or more embodiments, the descriptor sets were analyzed to determine if the reasons driving a customer support request were identified by the descriptor set subject identifications.

The software model may be evaluated according to three categories, including a “good match” where the support request reason(s) are identified by the top words in the subject vector (i.e., the words with the highest weight or frequency), a “moderate” match where the support request reason(s) are identified by the second tier of words in the subject vector (i.e., words six to ten), and a “poor” match where, for instance, the top words in a subject vector do not match or identify the reasons the support request was initiated.

Alternatively, instead of selecting a pre-determined number of communication elements, post-clustering concentration analysis can analyze the subject vectors to identify communication elements that are included in several subject vectors having a weight quantifier (e.g., a frequency) below a specified weight threshold level that are then removed from the subject vectors. In this manner, the subject vectors are refined to exclude content data less likely to be related to a given subject. To reduce an effect of spam, the subject vectors may be analyzed, such that if one subject vector is determined to include communication elements that are rarely used in other subject vectors, then the communication elements are marked as having a poor subject correlation and is removed from the subject vector.

In another embodiment, the concentration analysis is performed on unclassified content data by mapping the communication elements within the content data to integer values. The content data is thus turned into a bag-of-words that includes integer values and the number of times the integers occur in content data. The bag-of-words is turned into a unit vector, where all the occurrences are normalized to the overall length. The unit vector may be compared to other subject vectors produced from an analysis of content data by taking the dot product of the two-unit vectors. All the dot products for all vectors in a given subject are added together to provide a weighting quantifier or score for the given subject identification, which is taken as subject weighting data. A similar analysis can be performed on vectors created through other processing, such as K-means clustering or techniques that generate vectors where each word in the vector is replaced with a probability that the word represents a subject identification or request driver data.

To illustrate generating subject weighting data, for any given subject there may be numerous subject vectors. Assume that for most of subject vectors, the dot product will be close to zero—even if the given content data addresses the subject at issue. Since there are some subjects with numerous subject vectors, there may be numerous small dot products that are added together to provide a significant score. Put another way, the particular subject is addressed consistently throughout a document, several documents, sessions of the content data, and the recurrence of the carries significant weight.

In another embodiment, a predetermined threshold may be applied where any dot product that has a value less than the threshold is ignored and only stronger dot products above the threshold are summed for the score. In another embodiment, this threshold may be empirically verified against a training data set to provide a more accurate subject analysis.

In another example, a number of subject identifications may be substantially different, with some subjects having orders of magnitude fewer subject vectors than do other subjects. The weight scoring might significantly favor relatively unimportant subjects that occur frequently in the content data. To address this problem, a linear scaling on the dot product scoring based on the number of subject vectors is applied. The result provides a correction to the score so that important but less common subjects are weighed more heavily.

Once all scores are calculated for all subjects, then subjects may be sorted, and the most probable subjects are returned. The resulting output provides an array of subjects and strengths. In another embodiment, hashes may be used to store the subject vectors to provide a simple lookup of text data (e.g., words and phrases) and strengths. The one or more subject vectors can be represented by hashes of words and strengths, or alternatively an ordered byte stream (e.g., an ordered byte stream of 4-byte integers, etc.) with another array of strengths (e.g., 4-byte floating-point strengths, etc.).

The interactive content software service can also use term frequency-inverse document frequency software processing techniques to vectorize the content data and generating weighting data that weight words or particular subjects. The tf-idf is represented by a statistical value that increases proportionally to the number of times a word appears in the content data. This frequency is offset by the number of separate content data instances that contain the word, which adjusts for the fact that some words appear more frequently in general across multiple shared experiences or content data files. The result is a weight in favor of words or terms more likely to be important within the content data, which in turn can be used to weigh some subjects more heavily in importance than others. To illustrate with a simplified example, the tf-idf might indicate that the term “password” carries significant weight within content data. To the extent any of the subjects identified by a natural language processing analysis include the term “password,” that subject can be assigned more weight by the interactive content software service.

The content data can be visualized and subject to a reduction into two-dimensional data using a UMAP to generate a cluster graph visualizing a plurality of clusters. The interactive content software service feeds the two-dimensional data into a DBSCAN and identify a center of each cluster of the plurality of clusters. The process may, using the two dimensional data from the UMAP and the center of each cluster from the DBSCAN, apply a KNN to identify data points closest to the center of each cluster and shade each of the data points to graphically identify each cluster of the plurality of clusters. The processor may illustrate a graph on the display representative of the data points that are shaded following application of the KNN.

The system can also incorporate Part of Speech (“POS”) tagging software code that assigns words a part of speech depending upon the neighboring words, such as tagging words as a noun, pronoun, verb, adverb, adjective, conjunction, preposition, or other relevant parts of speech. The interactive content software service can utilize the POS tagged words to help identify questions and subjects according to pre-defined rules, such as recognizing that the word “what” followed by a verb is also more likely to be a question than the word “what” followed by a preposition or pronoun (e.g., “What is this?” versus “What he wants is an answer.”).

POS tagging in conjunction with Named Entity Recognition (“NER”) software processing techniques can be used by the interactive content software service to identify various content sources within the content data. NER techniques are utilized to classify a given word into a category, such as a person, product, organization, or location. Using POS and NER techniques to process the content data allow the system to identify particular words and text as a noun and as representing a person participating in the discussion (i.e., a content source). Recognizing participants in content data is also called diarization and is discussed more fully below.

### Large Language Models

Natural language processing can be implemented by a LLM, which is a sophisticated artificial intelligence model that is configured specifically for natural language processing tasks. Large language models are designed to understand and generate human-like text based on the patterns and structures that the LLM learns from processing substantial volumes of training data.

Large language models are implemented with a deep learning architecture called a transformer. Transformers consist of multiple layers of self-attention mechanisms that allow a LLM to weigh the importance of different words or tokens in a sequence and capture the relationships between them. The attention mechanism allows LLM to effectively process and generate text with contextually relevant and coherent patterns. By assigning different weights to different words, LLMs can effectively focus on the most relevant information, which facilitates accurate and contextually appropriate content generation.

Large language models learn by predicting the next word in a given context using an unsupervised learning process. Through repetition and exposure to training data, the LLM develops a proficiency for grammar, semantics, and the knowledge contained in the training data. During a pre-training phase, training data is ingested by a LLM and tokenized to break the training data down into smaller units called tokens. Tokens can be words, subwords, or characters. Tokenization allows a LLM to process and understand text at a granular level. The LLM learns to predict the next token in a sequence, given the preceding tokens.

After pre-training, a LLM can be fine tuned and applied to a wide range of applications that entail performing specific tasks, such as sentiment analysis, diarization, a chat bot, virtual assistant, or a content generation system. Fine-tuning involves providing the LLM with task-specific labeled data, so the LLM learns intricacies of a particular task.

The LLM can be used for inference after being trained and fine tuned. Inference uses the LLM to generate text or perform specific language-related tasks. During inference, LLMs employ a beam search technique to generate the most likely sequence of tokens. The beam search algorithm explores several possible paths in the sequence generation process while keeping track of the most likely candidates based on a scoring mechanism. This approach helps generate more coherent and high-quality text outputs.

There are various types of LLMs, including, without limitation: (i) autoregressive language models; (ii) encoder-decoder models; (iii) transformer-based models; (iv) trained and fine-tuned models; and (v) hybrid models. Autoregressive models generate text by predicting the next word given the preceding words in a sequence. Encoder-decoder models are commonly used for machine translation, summarization, and question-answering tasks. Encoder-decoder models consist of two main components: (i) an encoder for processing input sequences; and (ii) a decoder that generates the output sequence. A transformer LLM is a type of encoder-decoder architecture.

Hybrid LLMs combine the strengths of different architectures. For example, some LLMs may incorporate both transformer-based architectures and recurrent neural networks. RNNs are commonly used for sequential data processing and can be integrated into a LLM to capture sequential dependencies in addition to the self-attention mechanisms of transformers.

### Smart Research Design Assistant

The present invention is a computer-implemented system that assists researchers in designing immunofluorescence-based cellular experiments using a graphical user interface (“GUI”) enhanced by artificial intelligence (“AI”) and machine learning (“ML”). The system methods also provide hypothesis on protein interaction. The system relies on AI technology and LLMs that allow an enterprise to increase efficiency by reducing trial-and-error in experiment design, improve accuracy by leveraging verified data and predictive modeling, providing scalability by supporting single and multiplexed protein studies, and providing nuanced insights by enhancing understanding from molecular weight to three dimensional spatial interactions.

The system is configured to analyze user experiment inquiries such that the system can be efficiently and expediently scaled to handle larger volumes of experiment inquiries without committing the substantial degree of system and personnel resources required by conventional design techniques.

The smart research design assistant (also referred to as “design assistant”) is an artificial intelligence driven software program that interfaces with end users via natural language input through a web app, webpage, mobile software application, or similar interactive format. The design assistant enables researchers to design complex biological imaging experiments by inputting a desired molecular pathway of interest, up to three target proteins, primary and secondary antibodies, fluorophores, and optical system specifications. The system dynamically filters and recommends compatible components and protocols using assay function data. The smart research design assistant is trained using a corpus of data that includes, but is not limited to, compatibility of target proteins with primary and secondary antibodies, antibody-fluorophore compatibility data, experimental protocols, imaging parameters, pathway interdependencies, and historical usage data, such as input reflecting user research objectives, experimental hypotheses, imaging strategies and settings, post processing and pattern recognition, and imaging device selections to image particular proteins and achieve particular research objectives.

In one or more embodiments, the user inputs experimental parameters through an intuitive GUI that is rendered on a user computing device. The user accesses a provider or enterprise system that includes an external network computing device, such as a web server. In accessing the provider system, the user computing device transmits a user interface transmit command to the external server that can include: (i) an Internet Protocol (“IP”) address for the user computing device; and (ii) system configuration data (e.g., data identifying the operating system running the user computing device). In response to the user interface transmit command, the external server returns interface display data that is used by the user computing device to render one or more graphical user interfaces. After receiving interface display data, the user computing device processes the display data and renders GUI screens presented to users, such as a provider website or a GUI within a provider mobile software application.

The interface display data can include one or more of the following: (i) webpage data used by the user computing device to render a webpage in an Internet browser software application; (ii) mobile app display data used by the user computing device to render GUI screens within a mobile software application; and (iii) content data such as text and images to display in a graphical user interface. The interface display data can be in JSON or markup language format and include data that is used by the user computing device to format a GUI, such as data specifying the sizing and position of frames, data fields, and other inputs and display elements. Categories of interface display data can include graphical elements, digital images, text, numbers, colors, fonts, or layout data representing the orientation and arrangement graphical elements and alphanumeric data on a user interface screen.

Once experiment parameters are entered, the system filters options based on compatibility and prior experimental success. Inputs may include molecular pathway of interest, target proteins, antibodies, fluorophores, and optical systems (e.g., confocal, STORM, PALM). The system is able to dynamically filter and predict options for the experimental design. Based on the selected optical system, the smart research design assistant filters out incompatible fluorophores and antibodies, displays only relevant testing protocols, suggests experimentally verified combinations, and predicts likely successful combinations when no prior data exists.

The optical system input by the user can include a variety of different optical imaging systems. Some examples include: widefield microscopy, structured illumination microscopy, confocal microscopy, and super-resolution microscopy. Super-resolution microscopy systems can include Airyscan, super-resolution SIM, super-resolution SIM2, stochastic optical reconstruction microscopy (“STORM”), super-resolution photoactivated localization microscopy (“PALM”), and others. Generally, these optical systems can utilize light provided by light emitting diodes (“LEDs”), lasers, or other light sources. The optical systems can also include detectors such as cameras, photomultiplier tubes, confocal pinholes, CMOS sensors, and others. Such details can be input by the user.

Non-microscopic visualization methods can also be included, such as western blotting. Each system provides differing levels of resolution and can provide different types of useful information. For example, a user would know via western blot data that the protein targeted antibody is the correct molecular weight(s). Microscopy in the standard resolutions (above 200 nm), would provide localization information, such as cellular compartmentalization. In the case of manipulation re-localization, wherein the location or distribution of the studied protein is observed after the cellular environment is intentionally altered, this information also would be useful in calculating at standard resolutions the amount of protein that is present and changes to the protein accumulation or diminishment in the presence of manipulation.

When moving below standard resolutions (i.e., super resolution <220 nm), localization and correlative protein accumulation remain discernable along with the structure of specific targets, and importantly, it is possible to more accurately measure the localization of accumulation and/or recession of protein in the presence of manipulation. At single molecule resolution (i.e., <20 nm), protein structure is further enhanced to provide sub-unit structure, which conveys detailed information about the spatial organization of a specific single molecule of the protein target. At single molecule resolution, not only does 3-dimensional spatial information become available, but location mapping is enabled. At super resolution and particularly single molecule resolution, not only does three-dimensional spatial information become available, but location mapping is enabled.

One target can demonstrate all of the aforementioned. However, when comparing two or more targets simultaneously, spatial colocalization of the different protein molecules can be determined, and sub-unit interactions can be mapped with relative certainty. With this data, manipulation of the cellular environment by pharmaceuticals is highly effective and can be studied at the single molecule level. In short, the western blot is effective in that it provides the user a known weight, providing confidence in the targeting in the microscopy localization studies, and moving from standard resolution, to super resolution, to single molecule resolution allows assessment of protein function in orders of magnitude that coincide with the increase in resolution to the single molecule level.

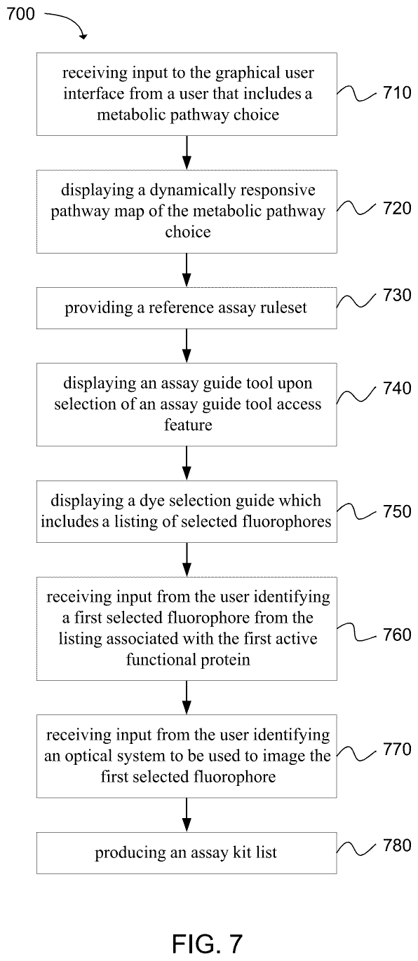

With this description in mind, FIG. 7 is a flowchart illustrating an example computer-implemented method for designing immunofluorescence cellular research tests in a graphical user interface 700. The method includes: receiving input to the graphical user interface from a user that includes a molecular pathway choice 710; displaying a dynamically responsive pathway map of the molecular pathway choice 720; providing assay function data 730; displaying an assay guide tool upon selection of an assay guide tool access feature 740; displaying a dye selection guide which includes a listing of selected fluorophores 750; receiving input from the user identifying a first selected fluorophore from the listing associated with the first active functional protein 760; receiving input from the user identifying an optical system to be used to image the first selected fluorophore 770; and producing an assay kit list 780.

The system further includes invoking a smart research design assistant that operates in conjunction with the graphical user interface to provide real-time, context-aware recommendations throughout the test design process. The smart research design assistant incorporates machine learning technology to analyze user inputs, historical assay data, and experimental constraints to suggest optimal antibody-fluorophore combinations, imaging parameters, and experimental protocols. The smart research design assistant can be configured to assist in hypothesis generation, experimental planning, and validation strategies, thereby enhancing the accuracy, reproducibility, and efficiency of the designed immunofluorescence tests.

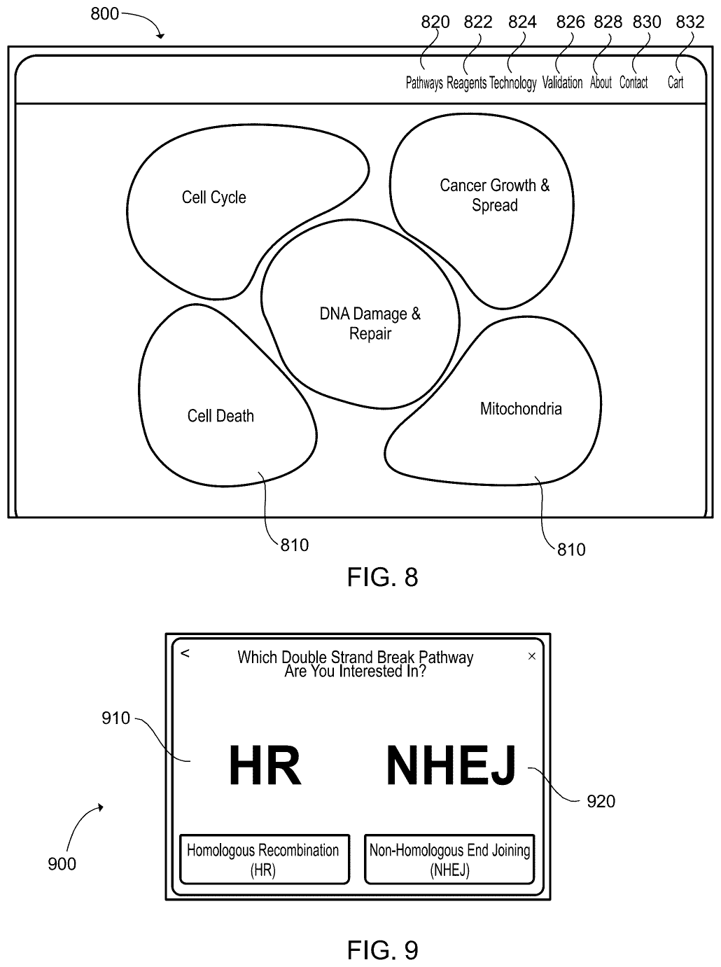

An example graphical user interface 800 is shown in FIG. 8. This graphical user interface can be used in the computer-implemented methods described herein. This graphical user interface includes multiple molecular pathways 810 that can be selected by the user. The molecular pathways in this example include: Cell Cycle; Cancer Growth & Spread; DNA Damage & Repair; Cell Death; and Mitochondria. When a user selects one of these pathways, the graphical user interface can display a dynamically responsive pathway map of that particular pathway. This GUI also includes links to graphical user interface displays for Pathways 820, Reagents 822, Technology 824, Validation 826, About 828, Contact 830, and Cart 832.

The smart research design assistant provides intelligent, context-aware support throughout the user's interaction. The smart research design assistant can proactively suggest relevant pathways and dynamically adapt sub-pathway suggestions based on the user's research objectives, prior selections, or experimental context reagents. The smart research and design assistant can also suggest imaging technologies based on prior selections, experimental goals, or historical usage patterns. It may also assist in refining experimental hypotheses, validating reagent compatibility, and optimizing imaging strategies, thereby streamlining the research design process and improving experimental reliability. As an example, a user can enter natural language prompts asking the smart research design assistant to make hypotheses regarding whether specified reagents will interact with specified proteins as part of a cellular pathway. The user can then ask the smart research design assistant to generate molecular experiment protocols that will test the reagent interactions with the specified proteins.

The smart assistant can further be configured to recommend optimal entry points for hypothesis testing (i.e., strategically valuable proteins or molecular interactions within a biological pathway that a research may focus their experiment on because these points are highly relevant, well studied, influential (e.g., upstream regulators), and technically feasible to target based on available antibodies and imaging tool), and provide contextual insights into pathway interdependencies (i.e., the functional relationships and interactions between different proteins within a biological pathway), thereby enhancing the user's ability to design targeted and efficient experiments. In this way, the smart assistant may provide a starting place for a user's research. For example, when studying DNA damage response, the smart assistant may recommend starting with RAD51 because it is involved early on in the pathway and provides measurable signals. In another example, when studying a DNA damage repair pathway, the smart assist may recommend ATM to study upstream regulation of p53 because ATM kinase activates CHK2, which in turn regulates p53.

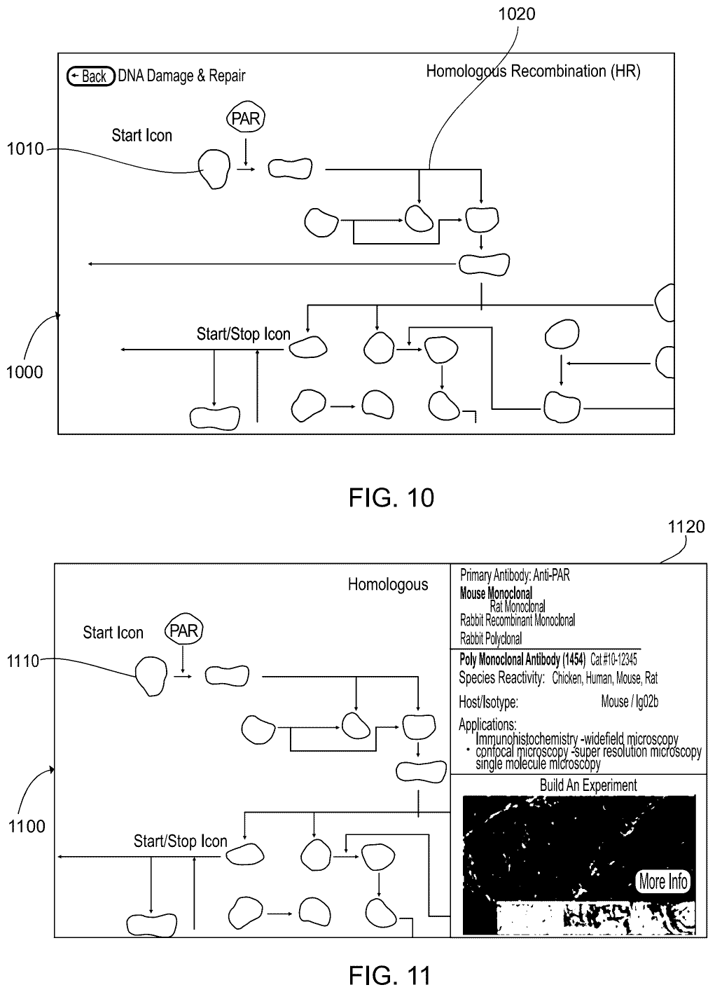

One or more of these pathways can have multiple sub-pathways. When the user selects a pathway that has multiple sub-pathways, then the graphical user interface can display a secondary selection of the sub-pathways. The user can then select the desired sub-pathway, and then a dynamically responsive pathway map will be displayed for that sub-pathway. As an example, the pathway for DNA damage and repair can include a first sub-pathway for homologous recombination double strand break, a second sub-pathway for non-homologous end joining (“NHEJ”) double strand break, and a third sub-pathway for single strand break. In certain examples, the sub-pathways can be displayed in one or more full screen displays, pop-up windows, pop-up menus, drop-down menus, or other display methods. An example pop-up window 900 is shown in FIG. 9. This pop-up window includes selections for the Homologous Recombination (“HR”) sub-pathway 910 and the Non-Homologous End Joining (“NHEJ”) sub-pathway 920. This window can be displayed after a user selects DNA Damage and Repair in some examples.

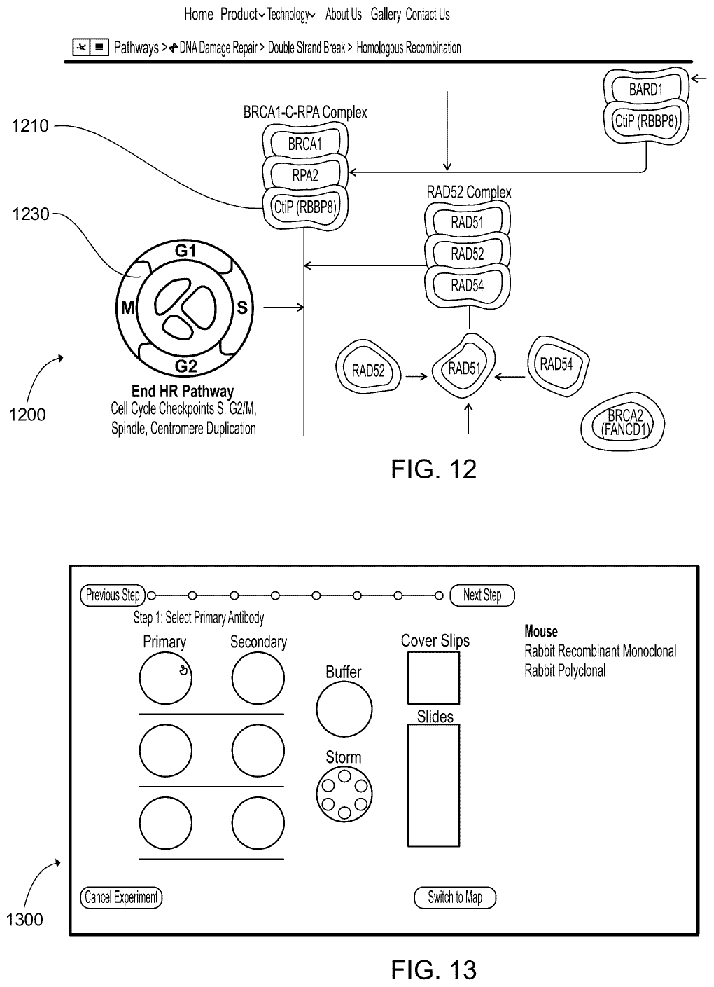

FIG. 10 shows a portion of a dynamically responsive pathway map 1000. A user can navigate to view other portions of the pathway map using any suitable navigation method, such as click and drag, scroll bars, finger gestures, and so on, depending on the particular user interface device (i.e., tablet, desktop, smartphone, etc.). This dynamically responsive pathway map includes many active functional proteins 1010 reactively connected with one another by arrows 1020 to form the dynamically responsive pathway map. The dynamically responsive pathway map can include all proteins involved in the particular molecular pathway as well as a portion of the proteins involved in the pathway. The pathway map is generated or refined by the smart research design assistant, which dynamically constructs the map based on the selected pathway, experimental objectives, and available data. The smart research design assistant prioritizes relevant protein interactions, highlights key nodes based on user-defined hypotheses, and adapts the map structure in real time to support iterative experimental planning.

In one example, the molecular pathway choice can be DNA damage and repair and the dynamically responsive pathway map can be a homologous recombination double strand break pathway. The portion of the active functional proteins displayed in this pathway map can include one or more of: Poly (ADP-ribose) polymerase 1 (PARP1), MRE11, RAD50, Nijmegen breakage syndrome 1 (NBS1), Replication Protein A (RPA), Bloom syndrome protein (BLM), Breast cancer 1 (BRCA1), CtBP Interacting Protein or RB Binding Protein 8 (CtIP), RAD51, RAD52, RAD54, FANCN, BRCA2, DSS1, FANCJ (BRIP1 or BACH1), ataxia-telangiectasia mutated (ATM), Tat-interactive protein, 60 kDa (TIP60), Checkpoint kinase 2 (CHK2), BRCA1-Associated RING Domain protein 1 (BARD1), HERC2, H2AX, MDC1, Histone protein 2A (H2A), RNF168, UBC13, Mms2, RNF8, P53-binding protein 1 (53BP1), deubiquitinase DUB (USP28), P53, GADD45, cyclin-dependent kinase inhibitor 1A (aCDKNIA), CIP1, Ccdc98, BRCC36, BRCC45, SUMO.Ub, Small Ubiquitin-like Modifier (SUMO), RNF4, MDC1, and UBC13.

In another example, the molecular pathway selection can be DNA damage and repair, and the dynamically responsive pathway map is a non-homologous end joining (NHEJ) double strand break pathway. The active functional proteins displayed in this pathway map include one or more of: P53-binding protein 1 (53BP1), ataxia-telangiectasia mutated (ATM), PTIP, 53BP1, artemis (SNMIC), ASFI, Rap1-interacting factor 1 (RIF1), mitotic arrest-deficient (Mad2L2/REV7), TRIP13, Ku70, Ku80, and DNA-Pkcs.

The dynamically responsive pathway map can also include information callouts associated with at least a portion of the active functional proteins. The information callouts can display a functional description of the active functional proteins and be configured as a pop-up window, a full-screen display, a text box, tool tip, text bubble, or other format. The information callouts are hidden in an initial view of the dynamically responsive pathway map, and a user selects one or more of the active functional proteins to display the information callouts for those proteins. The user can also select a protein using an action such as mouse clicking, mouse double clicking, mouse hovering, tapping, long pressing, or another selection method.

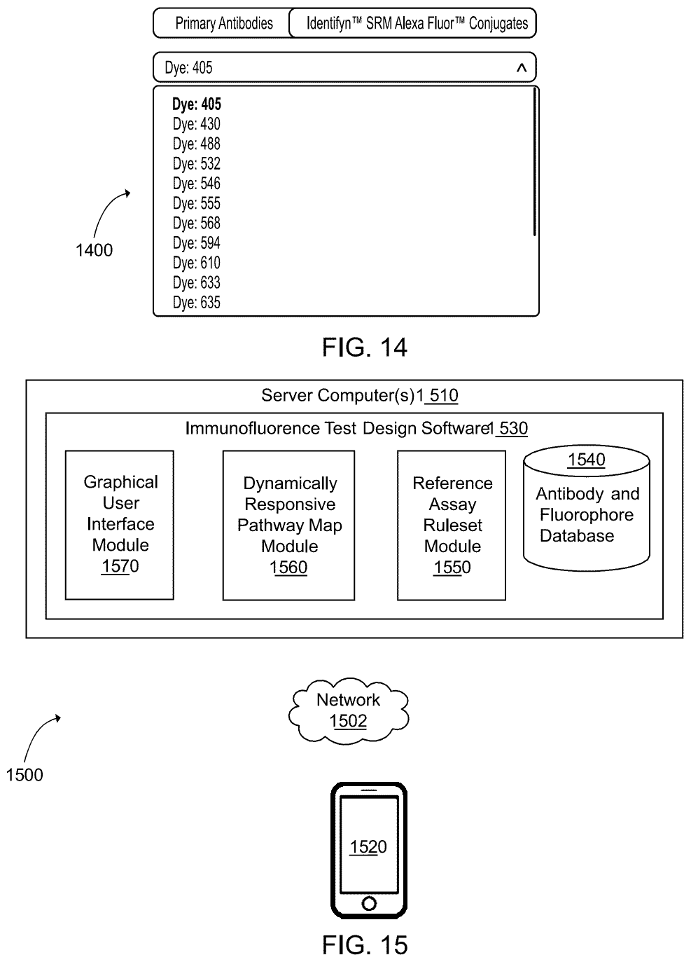

FIG. 11 shows an example dynamically responsive pathway map 1100 that includes active functional proteins 1110 and an information callout 1120 displaying a functional description of a selected protein. In this example, the information callout is a pop-up window or frame that appears to one side of the graphical user interface when the user selects a protein. The information displayed in this information callout includes primary antibodies, with host species and type. In this case, the primary antibody is Anti-PAR and the host species and types listed included mouse monoclonal, rat monoclonal, rabbit recombinant monoclonal, and rabbit polyclonal. The information callout also includes more detailed information about the mouse monoclonal antibody, including the species reactivity, host and isotype, applications of the antibody, concentration of the antibody available for purchase, and the price to purchase the antibody directly through the graphical user interface. The information callout also includes a link to add the antibody to a virtual shopping cart and a “buy now” link that the user can use to easily purchase the antibody.

The functional descriptions of the proteins include information about the function of proteins in a cell based on published literature or other verified sources. As an example, an information callout for the protein Rad51 can include information about the role of Rad51 in the homologous recombination process in repairing DNA double-strand breaks. The information callout can also display information about how Rad51 can be studied via immunofluorescence. The information callout provides information about the role of Rad51 in the particular pathway shown in the dynamically responsive pathway map, as well as in other cellular pathways. Such descriptions aid researchers in choosing protein targets to confirm accuracy of the pathway map, identify drug candidates, and the like. The information callout can also include a summary of the role of the Rad51 protein. These same points of information are displayed in information callouts for other proteins, among a variety of other types of information that may be useful to a user. The information callouts can also include reference citations to sources where the information originated. In certain examples, the citations can include links to access the original sources.

The information displayed in the dynamically responsive pathway map can be updated based on research published over time. Some users may be aware of new research that has not been cited or included in the dynamically responsive pathway map. Therefore, the GUI provides a user input option to allow users to suggest revisions to the functional descriptions of proteins, the dynamically responsive pathway map, or any other information displayed by the system.

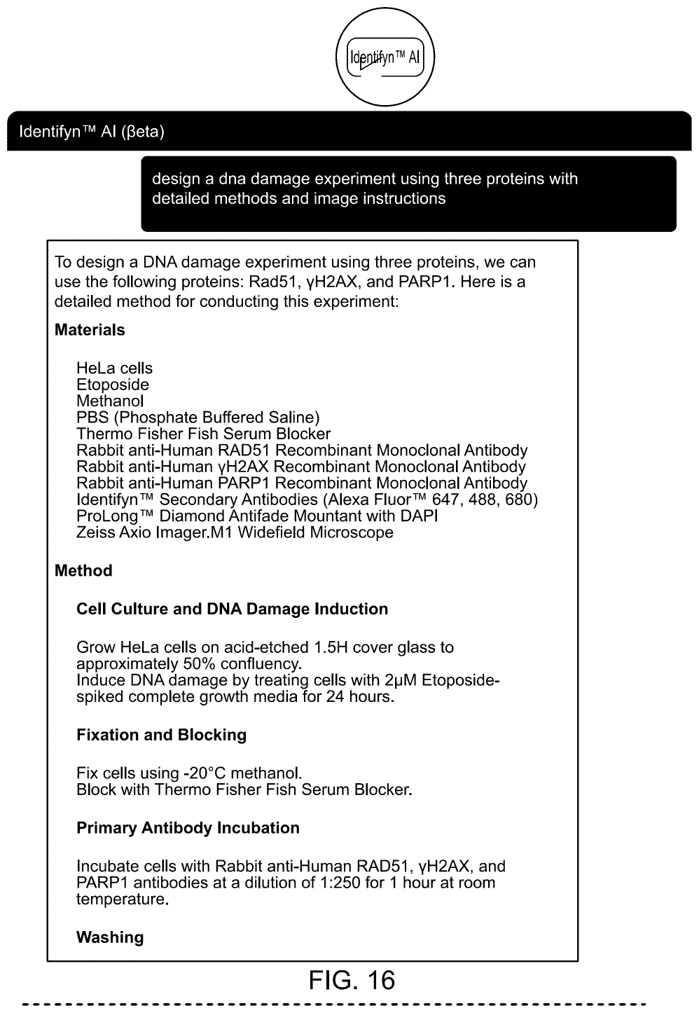

Another example display of a dynamically responsive pathway map 1200 is shown in FIG. 12. This pathway map includes active functional proteins 610 reactively connected with one another by arrows as in the previous example. This example also shows an end symbol 1230 representing an end of the pathway. The dynamically responsive pathway map can include one or more symbols (i.e., graphical icons) that are indicative of cellular choice. The icons may lead to the conclusion of the action of the pathway, such as a return to cell cycles of normal function and continued propagation in the expected manner. In some cases the pathway is ineffective, and the process is taken over by other cellular health pathways, and in the final case, the cell may determine that programmed cellular death is necessary (i.e., apoptosis). The end point of each pathway is a bifurcation to an additional pathway, or cellular death.

The dynamically responsive pathway map includes an assay guide tool access feature. The assay guide tool access feature can include a link, button, icon, or other input function that can be selected by a user. The assay guide tool is displayed when an end user selects the assay guide tool function. The assay guide tool can be displayed for up to three active functional proteins. The assay guide tool provides primary antibody selection options for each of the active functional proteins. The primary antibody selections can be filtered using the assay function data. For example, if a user selected multiple active functional proteins, the user can select a first primary antibody for a first protein; the user can then select a second primary antibody for a second protein from a listing of antibodies that has been filtered according to the assay function data and the first selected primary antibody.

The ruleset can include a variety of filtering rules, such as filtering the antibody selections to ensure that the second primary antibody is compatible with the use of the first primary antibody and the first protein. The filtering can include identifying cross-reactions. Similarly, if the user selects a third primary antibody for a third protein, then the ruleset can be used to filter a listing of primary antibodies from which the third primary antibody is selected. In various examples, the primary antibodies can be selected from full-length antibodies and antibody fragments (Fab).

The assay guide tool can be enhanced by the smart research design assistant, which automatically suggests optimal antibody combinations based on the selected proteins, experimental goals, and historical assay performance. The smart research design assistant may also provide real-time validation of antibody compatibility, highlight potential cross-reactivity risks, and recommend alternative reagents or configurations to improve assay robustness and reproducibility.

The assay guide tool provides a secondary antibody selection option for each of the up to three selected active functional proteins. The first secondary antibody selection option can be filtered using the assay function data to remove host and target species reactions with the first primary antibody selection option. The second active functional protein can be selected, and a second secondary antibody selection option can be filtered using the assay function data. A third active functional protein can also be selected a third secondary antibody selection option can be filtered using the assay function data.

The secondary antibodies can be useful when designing an experiment that utilizes secondary, or indirect, immunofluorescence. In such experiments, the second antibodies are conjugated to fluorophores for visualizing the proteins. In other experiments that utilize primary, or direct, immunofluorescence, the primary antibodies can be conjugated to fluorophores. Therefore, secondary antibodies may not be needed in such experiments. In various examples, there can be multiple proteins that are all visualized using direct immunofluorescence, or multiple proteins that are all visualized using indirect immunofluorescence, or multiple proteins where a portion are visualized using direct and a portion are visualized using indirect immunofluorescence.

The smart research design assistant can be configured to automatically recommend appropriate secondary antibodies based on the selected primary antibodies, species compatibility, and fluorophore requirements. The smart research design assistant may also flag potential conflicts, suggest optimized combinations for multiplexing, and adapt recommendations in real time as the user modifies experimental parameters.

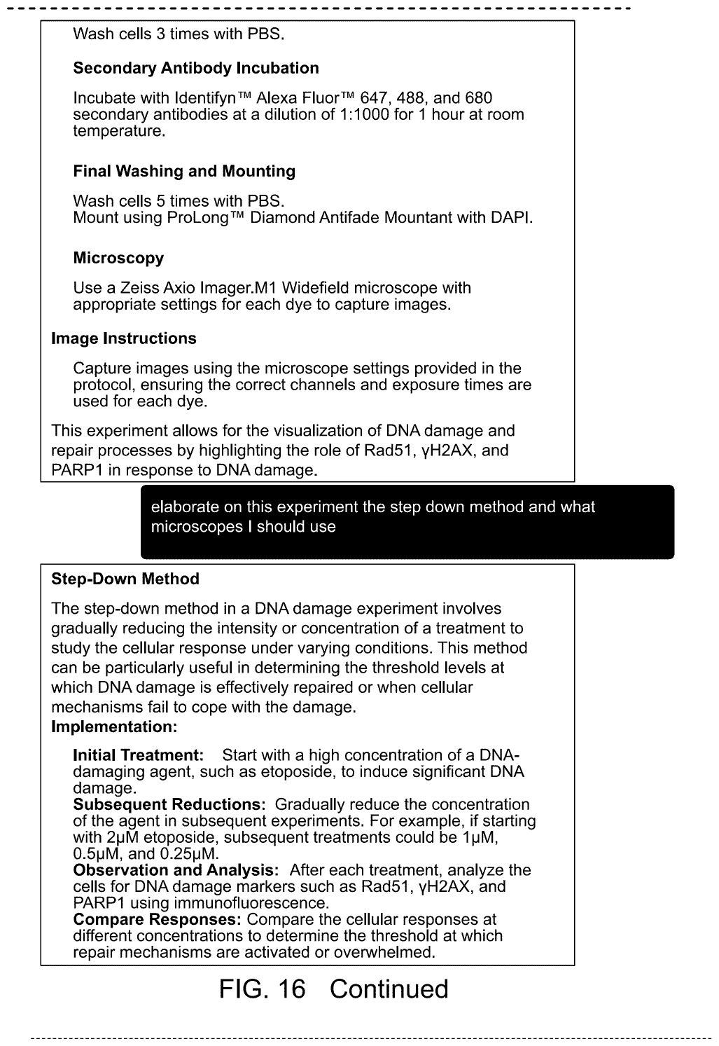

FIG. 13 shows an example display of an assay guide tool 1300. This assay guide tool includes selection choices for a first primary antibody, a first secondary antibody, a second primary antibody, a second secondary antibody, a third primary antibody, and a third secondary antibody. This assay guide tool also includes selection choices for a buffer, cover slips, slides, and STORM. The choice of proper reagents in providing increased (e.g., in some cases maximum) recovery of signal from within the cellular environment is important and can begin with the choice of proper refractive index (e.g., glass, coverslips, chambers, and other carrying cellular devices that are compatible with the optical information). The choice of mounting media is also a factor in ensuring a refractive index match to the choice of glass, further, in the case of STORM microscopy, the ability to induce “blinking” of the fluorophore is a factor while the rate, and duration of this phenomenon is critical to collecting and assembling image data. All told, the combination of glass, buffers, and non-cross reactive fluorophores contribute to image confidence.

The smart research design assistant be configured to optimize the foregoing selections by analyzing the user's experimental configuration and recommending compatible reagents, substrates, and imaging conditions. The smart research design assistant may also simulate expected signal performance based on selected parameters, helping users refine their setup for maximum imaging fidelity.

The assay function data can include information to be used in filtering antibodies and fluorophores and assisting a user in selecting appropriate antibodies and fluorophores for use with active functional proteins. The ruleset can also include additional information, such as recommendations for optical system settings and testing protocols. In some examples, the assay function data can include a database of antibodies corresponding to the portion of the active functional proteins. The database can also include fluorophores. The assay function data can correlate compatible antibodies and fluorophores with each of the active functional proteins. The database can also include antibody data which includes a host species and a reactive species and cross-reactivity information, and fluorophore data which includes fluorophore excitation and emission spectra to enable selection of fluorophores having spectral separation.

In one or more embodiments, the smart research design assistant interfaces with the assay function data to provide intelligent filtering, predictive compatibility scoring, and real-time feedback on reagent selection. This integration enables a more adaptive and guided design process, reducing the likelihood of experimental failure due to incompatible or suboptimal reagent combinations.

In various examples, the assay function data can include any of the following conditions or a combination thereof: a first primary antibody binds with the first active functional protein; a first secondary antibody binds with the first primary antibody; a species reactivity of the first primary antibody matches a species of the first active functional protein; a species reactivity of the first secondary antibody matches a first host of the first primary antibody; a second primary antibody binds with the second active functional protein and is produced from a second host which is not common with the first host; a second secondary antibody binds with the second primary antibody; a third primary antibody binds with the third active functional protein and is produced from a third host which is not common with the first host or the second host; a third secondary antibody binds with the third primary antibody; a first fluorophore binds to one of the first primary and secondary antibodies and has a first fluorescence response; a second fluorophore binds to one of the second primary and secondary antibodies and has a second fluorescence response, wherein the first fluorescence response is spectrally separated from the second fluorescence response; and a third fluorophore binds to one of the third primary and secondary antibodies and has a third fluorescence response, wherein the third fluorescence response is spectrally separated from the first fluorescence response and from the second florescence response.

The GUI also allows the user to input an optical system that is to be used to image the proteins. In some examples, the optical system can include western blotting, widefield microscopy, structured illumination microscopy, widefield structured illumination microscopy, confocal laser scanning microscopy, super resolution structured illumination microscopy, stochastic optical reconstruction microscopy, or combinations thereof. The computer-implemented methods described herein can also include adjusting the assay function data based on the user input optical system. The ruleset can be adjusted to filter out fluorophore candidates that would provide deleterious feedback and skew the experiment. The ruleset can also be adjusted to provide helpful testing protocols and optical system setting recommendations for the user that are specific to the type of optical system to be used.

The GUI optionally includes a dye selection guide that includes a listing of selected fluorophores. The dye selection guide can be displayed to allow the user to select dyes (i.e., fluorophores) that can be conjugated to either the primary antibodies or secondary antibodies. The dye selection guide can display a listing of fluorophores produced from a fluorophore catalog by filtering fluorophores using the assay function data to list suitable fluorophores for each of the selected active functional proteins. In some examples, the dye selection guide can allow the user to select a wavelength of the fluorophore, which can include the emission wavelength, excitation wavelength, or both. After the user has selected a wavelength, the dye selection guide can display a listing of fluorophores that is filtered to comply with the wavelength selection and with the assay function data. In further examples, the dye selection guide can display a graphical fluorescence curve for a plurality of spectral bandwidths of various fluorophores.

The smart research design assistant can enhance the dye selection process by automatically recommending fluorophores based on spectral compatibility, antibody conjugation preferences, and the user's selected imaging system. The smart research design assistant may also simulate spectral overlap, suggest multiplexing strategies, and provide real-time feedback to ensure optimal signal separation and imaging performance.

Once a dye is selected, the smart research design assistant and provide instruction for sample preparation to achieve optimal dye saturation as part of a molecular experiment protocol generated and displayed to the user. The instructions can include, for instance, specific recommended steps and materials used to prepare a buffer, prepare a dye stock, perform a buffer exchange, and perform dye conjugation.

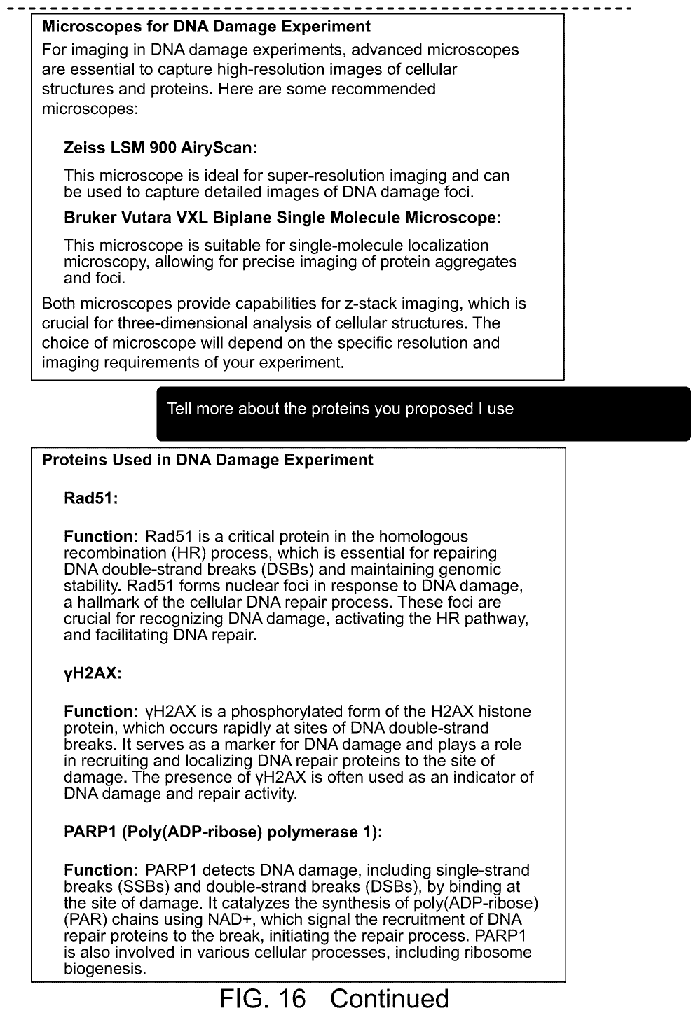

FIG. 14 shows an example dye selection guide 1400. This example includes a drop-down box with a listing of dyes having different emission wavelengths. The number of each dye corresponds to the peak emission wavelength of the dye. The user can select any of the listed dyes.

In various examples, the listing of selected fluorophores in the dye selection guide can be filtered using one or more of the following parameters: color of the fluorophore; when the fluorophore is conjugated to an antibody, the host species of the antibody; when the fluorophore is conjugated to an antibody, the reactivity of the antibody; when the fluorophore is conjugated to an antibody, the cross species reactivity of the antibody; whether or not the fluorophore is a secondary antibody conjugate; whether or not the fluorophore is a primary antibody conjugate; and the optical system to be used to image the fluorophore. Dye selection is an important factor to image acquisition, and optimally selected dyes will show no overlap in the excitation and emission spectra. However, when overlap is unavoidable, adjustment can be suggested to tune the instrumentation collection parameters to match that of the dyes chosen.

The system can also include producing an assay list (also referred to herein as a materials list or a list of material identifications), which includes a multitude of selected active functional proteins, selected primary antibodies, and selected fluorophores, as well as recommended testing protocols. Those of skill in the art will appreciate that any number of proteins, antibodies, and fluorophores can be used that a user's particular application can accommodate spectrally. Any additional antibodies and fluorophores that were selected by the user can also be included in the assay list. In further examples, the assay list can also include any additional reagents or materials that are to be used in the experiment designed by the user. In certain examples, the recommended testing protocols can include a recommended order for reacting the first primary antibody, the first selected fluorophore, and additional primary antibodies, fluorophores, or secondary antibodies if present in the assay list. In further examples, the recommended testing protocols can include optical limitations of the first selected fluorophore and any additional fluorophores if present in the assay list. In still further examples, the recommended testing protocols can include testing protocols for performing a control experiment.

The smart research design assistant can automatically generate the assay list by synthesizing user selections, experimental goals, and compatibility data from the assay function data. The smart research design assistant may also tailor the recommended testing protocols based on the selected optical system, suggest control conditions, and flag any potential conflicts or missing components to ensure experimental completeness and reproducibility.

The assay list can include links to add individual items in the list to a virtual shopping cart to purchase the items. This can allow a user to easily purchase all the reagents and materials to be used in the designed experiment, or to produce a single kit having all of the items and including the test protocols as well as glassware used in an experiment. The test protocols can be provided to the user as part of a kit as a QR code (which links to a corresponding customized instruction set), printed instructions, custom hyperlink, etc.

The system includes a gallery that displays images generated using immunofluorescence. The images can be collected from published research or from other sources. The gallery GUI allows a user to upload images that the user has produced experimentally using immunofluorescence. The images in the gallery can serve as example images to illustrate results achieved by dying various proteins using antibodies and fluorophores. In addition to the images, the gallery can include a description associated with each image. The description can include detailed information on the proteins selected for imaging, the primary and secondary antibodies used to bind to the proteins, the fluorophores that were conjugated to the antibodies, the testing protocols that were used in performing the imaging, and the optical system used for the imaging. These details can be useful for users to help users reproduce all or a portion of the results shown in the images, perform analysis directly on such images. One can use the sample preparation methodology shown, and using the same system, or a similar system, work backward from the details provided and produce the exact same image with no need for learning, experimentation, or design.

The smart research design assistant can automatically generate image descriptions by analyzing the image metadata, associated assay parameters, and experimental context. The smart research design assistant can also infer missing details, standardize terminology, and cross-reference the image with known protocols and fluorophore profiles to ensure consistency and reproducibility across the gallery.

When a user uses the computer-implemented methods described herein, by designing a cellular test using the graphical user interface, the user is enabled via the computer-implemented method to make correct and informed choices as to the experimental design, products used such as antibodies and fluorophores, reactivity and spectral information of the antibodies and fluorophores, based on an expansive collection of data that can be contained in the assay function data. When the user inputs the optical system that the user intends to use for the test, the computer implement methods can tailor the information provided to the user based on the specific experiment and optical system. The computer-implemented methods can provide the user with optical settings that have already been tested to ensure success.

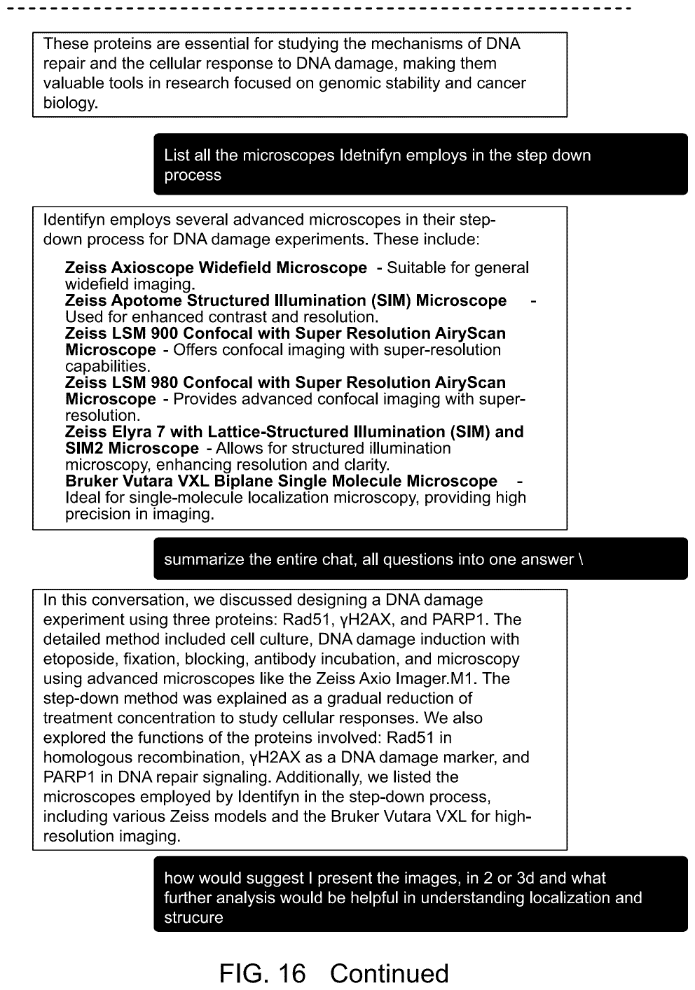

FIG. 15 illustrates a simplified example system 1500 for designing immunofluorescence cellular research tests in a graphical user interface. The system includes one or more server computers 1510 in signal communication with a user device 1520 through a network 1502. The server computer(s) runs immunofluorescence test design software 1530, which can be configured to allow the user to perform the computer-implemented methods described above. The software can also include a graphical user interface module 1570 configured to provide the graphical user interfaces described above.

### Example 1

In one example, a user selects DNA damage and repair: homologous recombination double strand break pathway from a listing of molecular pathways in a graphical user interface as described herein. The smart research design assistant then generates and displays via a GUI a dynamically responsive pathway map of the selected pathway. The user then selects Rad51, which is the user's primary protein of interest, from the dynamically responsive pathway map. The smart research design assistant then generates and displays via a GUI an information callout including information about the protein's role in the pathway and known protein-protein interactions based on information stored in the assay function data database. Based on this information, the user determines additional proteins that the user wishes to examine that may directly interact with Rad51 or be affected by Rad51 downstream of a molecular event, or conversely, chooses an upstream molecular event that may affect Rad51 response. Upstream, downstream, and direct interactions may be tested via manipulation of the cellular environment and the test design can be inherent in examination of the information displayed to the user by the graphical user interface.

The smart research design assistant then identifies and presents available primary antibodies that can bind to the selected protein targets within a cellular environment. The user can select a primary antibody that is unconjugated, which can be used together with a secondary antibody that is conjugated to a fluorophore. Alternatively, the user can select a primary antibody that is conjugated to a fluorophore, in which case no secondary antibody is used. If an unconjugated primary antibody is selected, the smart research design assistant automatically selects a compatible secondary antibody conjugated to a fluorophore, ensuring species specificity and avoiding cross-reactivity. This process can be repeated for up to three protein targets. If the smart research design assistant determines that cross-reactivity is unavoidable or acceptable for the experimental design, it provides a warning and justification for proceeding.

If a primary antibody conjugated to a fluorophore is selected, the design assistant bypasses the secondary antibody selection step and proceeds to configure the optical instrumentation parameters. It prompts the user to confirm or input specifications for the imaging system, such as microscope type, objective lens, and detection settings.

In cases where both primary and secondary antibodies are used and pass species compatibility checks, the smart research design assistant again requests or confirms the user's optical instrumentation specifications. These specifications are then used to guide fluorophore selection and imaging protocol optimization.

The user then chooses dye colors, multiplexing the dyes, and the smart research design assistant can provide direct feedback as to spectral separation. The smart research design assistant can indicate that the experiment design is specially correct and spectrally correct. The smart research design assistant provides a sort of “assay kit list” with a list of recommended materials to conduct a molecular experiment and recommended testing protocols to perform the experiment.

The system outputs can include a materials list, a molecular experiment protocol and a level of known confidence that the materials list and protocol will meet research objectives. The confidence level is determined using AI-based predictive modeling that evaluates the experimental configuration against a trained dataset of validated immunofluorescence assays. The smart research design assistant may use machine learning models (e.g., ensemble classifiers or neural networks) to assess the likelihood of successful signal separation, antibody compatibility, and imaging clarity based on parameters including fluorophore spectra, antibody host species, optical system specifications, and historical assay outcomes. The model outputs a confidence score, which reflects the predicted reliability of the experimental design. If the user has spectral overlap, then the smart research design assistant can offer alternative dyes to reach a level of separation deemed to provide the same aforementioned confidence. If the user has spectral overlap and it cannot be separated, the assistant can provide a confidence level, and introduce the possibility that the experimental design may be flawed and artificial data may be introduced. The user can be provided with test protocols specific to the user's experiment, with annotations indicating any limitations or risks identified by the smart research design assistant's analysis.

### Example 2

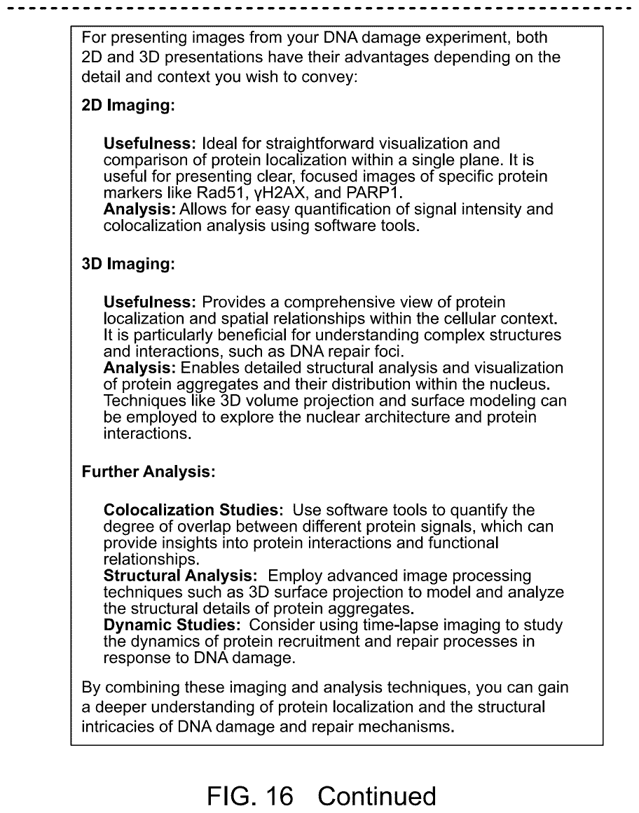

As shown in FIG. 16, a user enters a natural language prompt or any suitable alphanumeric text data input into a text box to communicate with the smart research design assistant. The input includes directions to design an experiment. The user directs the smart research design assistant to “design a dna damage experiment using three proteins with detailed methods and image instructions.” The design assistant responds by generating a detailed method for conducting the experiment. The detailed method may include materials (e.g., cells, proteins, buffer, primary antibodies, secondary antibodies, microscope), steps and methods to perform the experiment (e.g., cell culture and DNA damage induction; fixation and blocking; primary antibody incubation; washing; secondary antibody incubation; final washing and mounting; and microscopy details), imaging instructions (e.g., microscope settings), and an experiment summary.

The user may further direct the smart research design assistant to elaborate on the above-described experiment by inputting the prompt “elaborate on this experiment the step down method and what microscope I should use.” The smart research design assistant responds by generating a detailed step-down method that includes implementation (including initial treatment with an agent, subsequent reductions of the agent, observation and analysis of the cells, and comparison of cellular responses), recommended microscopes (e.g., Zeiss LSM 900 Airyscan, Bruker Vutara VXL Biplane Single Molecule Microscope).

The user may further direct the smart research design assistant to elaborate on the proteins to be used in the experiment generated by the smart research design assistant. For example, the user may put into the chat box “tell me more about the proteins you proposed I use.” The smart research design assistant responds by generating detailed descriptions for the proposed proteins. For the proteins Rad51, yH2AX, PARP1 (Poly (ADP-ribose) polymerase 1), for example, the smart research design assistant may provide information related to their functions, as shown in FIG. 16.

The user may further direct the smart research design assistant to elaborate on the all the microscopes Identifyn uses in its step-down process. For example, the user may put into the chat box “List all the microscopes Identifyn employs in the step down process.” The smart research design assistant responds by generating a list and descriptions of the microscopes used in the step-down process for DNA damage experiments (e.g., Zeiss Axioscope Widefield Microscope, Zeiss Aptome Structured Illumination (SIM) Microscope, Zeiss LSM 900 Confocal with Super Resolution AiryScan Microscope, Zeiss LSM 980 Confocal with Super Resolution AiryScan Microscope, Zeiss Elyra 7 with Lattice-Structure Illumination (SIM) and SIM2 Microscope, Bruker Vutara VXL Biplane Single Molecule Microscope). The description of the microscope may include information related to each microscope, the microscope capabilities, and the ideal imaging use of the microscope.

The user may further direct the smart research design assistant to summarize the entire conversation between user and the smart research design assistant. For example, the user may put into the chat box “summarize the entire chat, all questions into one answer.” The smart research design assistant responds by generating a summary of the entire conversation, as shown in FIG. 16.

The user may further direct the smart research design assistant to suggest how the images produced by the DNA damage experiment should be presented and what analysis would clarify location and structure. For example, the user may put into the chat box “how would suggest I present the images in 2 or 3d and what further analysis would be helpful in understanding localization and structure.” The smart research design assistant responds by generating information related to presenting images from the DNA damage experiment, including both 2D and 3D presentations. The information may include advantages of 2D and 3D presentations as well as each presentations usefulness and the analysis each provides. The smart research design assistant may also provide further analysis that includes software tools and imaging and analysis techniques that can be used to improve the user's experience.

### Post Processing and Image Analysis

Following experiment design, the user loads the materials to be imaged (e.g., the molecular target, the antibody, and the fluorophore) into the imaging device. The user operates the imaging device to generate microscopy image data that is used to render two- or three-dimensional images of the materials being imaged. Once an image is captured, the image data can be converted and/or stored in one or more suitable image data formats, such as a Joint Photographic Experts Group (“JPEG”) compliant format, a tabbed image file (“TIFF”) format, a bitmap format, or a Scalable Vector Graphics (“SVG”) image format. In some embodiments, the imaging device captures images in a first image data format, such JPEG, that is then converted to another format with a smaller file size to facilitate transmission of the image data between computing devices.

The imaging device is coupled to a computing device running the smart research design assistant. After generating the image data, the smart research design assistant can perform post-processing image analysis using the image data to verify the quality of the image and to verify that the image data actually represents the material that was imaged. The system uses image recognition and object detection software along with machine learning software to verify the image data. Image recognition software focuses on identifying what is depicted in an image and assigning a classification label to an image. Object detection software function by locating objects within the image using bounding boxes and erosions shells (see Bennett et al., H2AX chromatin structures and their response to DNA damage revealed by 4Pi microscopy, Proceedings of the National Academy of Sciences 2005, which is incorporated herein by reference). The system uses a combination of these software techniques, such as using object detection to locate an object within the image data before employing image recognition software to classify the object as a molecular target, antibody, fluorophore, or other material.

The machine learning software is implemented using one or more neural networks that are trained on existing, known image data. Common network architectures used for image recognition include convolution neural networks, support vector machines, and a histogram of oriented gradients architecture.

The image data is processed utilizing functions that include, without limitation: (i) a content recognition analysis; (ii) pattern recognition; (iii) template matching; (iv) feature recognition analysis to determine characteristics, such as physical dimensions, boundary edge locations, the location of known ligands or molecular elements; and (v) image enhancement operations, such as sharpening the image, de-skewing the image, de-speckling the image, reorienting the image, de-warping the image, converting the image to greyscale or black-and-white colorization (i.e., binarization), or adjusting the color.

To ensure human and machine readability of the image data, the system can perform one or more image enhancement operations. Enhancement operations include, but are not limited to, one or more of the following functions: (i) de-skewing an image where the edges of the imaging target material are rotated relative to the boundaries of the image (i.e., re-orienting the imaging target material to better align with the image boundaries); (ii) de-warping the image when the imaging target material is tilted (i.e., modifying portions of the imaging target material so that the imaging target material appears to be perpendicular to the camera lens); (iii) binarization to convert the image to black-and-white pixels; (iv) de-speckling to remove positive and negative spots and to smooth edges present in the image; (v) cropping pixels or portions of an image outside of the imaging target (i.e., the materials to be imaged); and (vi) down-sizing the image to a more suitable dots-per-square-inch (“DPI”) size that is more efficient to process and transmit over a network.

In some cases, the image data may include significant levels of noise that interferes with recognizing objects in the image. Noise reduction techniques can be applied to facilitate object recognition. Noise in digital images can include, without limitation, Gaussian noise, Rayleigh noise, salt and/or pepper noise, and impulse noise, among other types. Noise detection analyzes the color, brightness, or other properties of a pixel as compared to nearby pixels where pixels having characteristics that vary significantly compared to nearby pixels are taken as noise. The system can apply image quality thresholds, such as a specified noise ratio where images above the threshold are flagged as being poor quality and images below the threshold are accepted.

Noise reduction techniques can include running an edge adaptive spatial low pass filter over an image while using an edge detector to protect some of the edge boundaries. Another way to improve signal to noise ratios (“SNR”) is by temporally combining matching parts from two or more images by applying a temporal filter (e.g. a Motion Compensated Temporal Filtering). Gaussian noise reduction techniques can include mean filtering or Wiener filtering. Non-linear filters, such as median filtering and weighted median filtering, suppress noise without any identification. Bilateral filtering is a non-linear, edge-preserving, and noise-reducing smoothing technique that replaces the intensity value of each pixel with a weighted average of intensity values from nearby pixels. Spatial noise reduction techniques can include total variational regularization, non-local regularization, sparse representation, and low rand minimization techniques.

Transform domain de-noising techniques first transform the given noisy image to another domain, and then they apply a de-noising procedure on the transformed image according to the different characteristics of the image and its noise. Transform domain techniques include, without limitation, independent component analysis and MB3D. Neural networks can also be used for noise reduction where optimization techniques are employed, such as use of convolutional neural networks, multi-layer perception models, or deep learning networks.

The image data for the enhanced and de-noised image can be processed using object detection techniques that enable the smart research design assistant to recognize objects within the image data. Object detection techniques can utilize edge detection. To perform edge detection, the system first converts an image to black and white pixels with a “1” representing a non-white pixel and a “0” representing a white pixel and where each pixel has position data (e.g., X-Y coordinates) and a brightness value indicating how light or how dark the pixel is to be displayed. The system analyzes adjacent rows and columns of pixels to determine abrupt changes in the brightness values that represent edges of the material being imaged. The system can streamline the edge detection process by starting the analysis at locations where edges are expected.

Other object detection techniques include density-based clustering analyses. Clustering can also begin with binarization where each pixel is converted to black or white. The clustering algorithm identifies clusters of non-white pixels in an area of the image. Each separately identified cluster may be, for instance, a protein, antibody, or fluorosphere. The section of the image being examined is processed as a matrix of pixels where each non-white pixel is considered a data point for the clustering process.

One suitable density-based clustering algorithm is Density-Based Spatial Clustering of Applications with Noise (“DBScan”), which is a density-based clustering non-parametric algorithm. Given a set of points in a set space, the DBScan algorithm groups together pixels that are closely packed together (i.e., non-white pixels with many nearby neighbors that are also non-white pixels). The clusters packed together are recognized as objects in the image data. The algorithm also marks as outliers points that lie alone in low-density regions whose nearest neighbors are too far away (i.e., a pixel distance above a predetermined threshold). The output of the clustering algorithm is a dataset array that digitally identifies the X and Y and (voxels) coordinates of the pixels in each identified object cluster along with an assigned label for each cluster where the algorithm will assign the same cluster label to data points that are part of the same cluster.

The dataset array from the clustering algorithm is processed using cluster extraction and rescaling operation that extracts the individually identified clusters in the dataset array into individual dataset arrays and rescales each individual dataset array into, for example, a twenty-eight by twenty-eight (28×28) pixel cluster image, using extrapolation, which retains the main features of the image. The rescaling process also centers the cluster in the cluster image and adds border padding.

Once objects are detected within the image data, the system uses image recognition software to identify the detected objects. Image recognition software can use, for example, a classification model that employs a neural network, such as a convolutional neural network. The neural network includes an input layer that receives the cluster image, a convolutional layer that classifies the image, a pooling layer that reduces the dimensions of feature maps, a fully connected layer that connects the nodes between layers, and an output layer that outputs the classified objects in the image (e.g. the molecular target).

Image recognition software can utilize techniques such as color-based image recognition, template matching, or image segmentation and blob analysis. Color-based image recognition leverages the distinct spectral signatures of fluorophores that are commonly conjugated to antibodies targeting specific proteins. By isolating channels corresponding to particular wavelengths, color-based methods allow for the differentiation and quantification of multiple targets within a single sample of imaged material. In widefield or SIM images, where overlapping signals may occur, this technique is useful for detecting co-localization and spatial distribution of biomarkers.

Template matching relies on extraction of image features, such as shapes, textures, and colors that are compared against a reference image or template. At each position, a similarity score is calculated between the template and the overlapping region of the target image. Common similarity metrics include: (i) cross-correlation, which measures the linear relationship between the template and the image region where higher values indicate a stronger match; (ii) sum of squared differences that calculates the sum of squared differences between corresponding pixel values where lower values indicate a better match; and (iii) normalized cross-correlation, which is a normalized version of cross-correlation, robust to changes in illumination. Similarity scores above a specified threshold can be taken as a match.

Template matching is useful when protein or antibody-labeled features adopt predictable spatial patterns, such as the bands seen in western blots or repeated punctate structures in super-resolution images. By correlating predefined templates—based on known protein distributions or fluorophore patterns—with microscopy data, researchers can detect subtle structural elements that may be missed by simpler thresholding techniques. This is especially beneficial in super-resolution imaging (e.g., STORM or SIM) where spatial resolution is tens of nanometers, thereby revealing fine-grained subcellular architectures.

Image segmentation first partitions an image or object into multiple segments using techniques that include, without limitation: (i) thresholding, which separates pixels based on intensity values to create a binary image where objects are distinct from the background; or (ii) clustering algorithms, as discussed above, which groups pixels with similar characteristics. After segmentation, the system can apply a blob analysis where a blob is a connected region of pixels sharing a common property.

The system processes the blobs to extract various features that include, without limitation: (i) area—i.e., the number of pixels within the blob; (ii) the centroid, which is central point of the blob; (iii) the perimeter, which is length of the blob boundary; (iv) shape descriptors, which includes measures like circularity, aspect ratio, or moments that characterize the blob's shape; and (iv) orientation, which is the angle of the blob's major axis. The system compares characteristics of each blob and their relationships against known images to classify objects in the image.

In a simplified example, the system determines two-dimensional spatial distances between specified features in an image, and the spatial distance is compared to images of known molecular targets to identify a molecular target that has features substantially corresponding to the two-, three-, four-, or five-dimensional spatial distances between specified features in an image. The spatial distance is compared to images of known molecular targets to identify a molecular target that has features substantially corresponding to the two-, three-, four-, or five-dimensional spatial distance. In the context of microscopy, five-dimensional refers to imaging that captures data in five dimensions: the three spatial dimensions (x, y, z), wavelength (λ), and time (t). This allows for a more comprehensive understanding of dynamic processes within a sample, as it captures changes in both space and light properties over time.

The system applies the various image analysis software techniques to identify and classify the objects in the image data, and the results are rendered on a graphical user interface for display to the user. That is, the smart research design assistant analyzes the image and tells the user the types of molecular targets, antibodies, or fluorophores within the image. The smart research design assistant can also indicate to the user whether the image is good or poor quality and whether the identification of the materials using image recognition matches the expected identification of the materials that were input by the user.

The smart research design assistant is also configured to assist users throughout the process of image analysis. For instance, the smart research design assistant can recommend the image processing techniques that have the highest probability of meeting experiment objective and determining the information sought by the user, and the smart research design assistant provide instructions for using image recognition software to apply the recommended image processing techniques and desired post-processing views.

The recommendations for image processing are based on a wide range of inputs, such as localization, molecular sizing, and the presence of particular combinations in an experiment. For instance, if a user intends to conduct an experiment with multiple molecular targets (e.g., nine molecular targets in the image), the smart research design assistant can generate an experiment protocol having a specified order of taking various types of images while highlighting important colocalization events that should be observed.

The image processing recommendations and software instructions can be generated automatically or in response to a natural language prompt from a user, such as “please recommend image processing techniques for evaluating the amount of protein that is present and changes to the protein accumulation.”

Although the foregoing description provides embodiments of the invention by way of example, it is envisioned that other embodiments may perform similar functions and/or achieve similar results. Any and all such equivalent embodiments and examples are within the scope of the present invention.

What is claimed is:

1. A system for cellular experiments comprising:
(a) a computing device comprising a processor and an integrated display device;
(b) a machine-learning software module and training data, wherein the machine-learning software module causes the processor to iteratively train, using training data, a neural network by performing the operations of
(i) inserting the training data into an iterative training and testing loop to predict a target variable, wherein the target variable comprises a probability that a molecular experiment protocol meets experiment objective data,
(ii) repeatedly determining, during each iteration of the training and testing loop, the target variable, wherein each iteration of the training and testing loop has differing weights assigned to one or more nodes of the neural network, each of the differing weights being updated with each iteration of the training and testing loop to reduce error in predicting the target variable and improve predictive ability of the neural network, thereby creating a trained neural network, and
(iii) deploying the trained neural network;
(c) a memory device storing data and executable code that implements one or more integrated software applications that, when executed, cause the processor to:
(i) receive interface display data,
(ii) generate a graphical user interface using the interface display data, wherein the graphical user interface comprises an input field configured to receive natural language data,
(iii) render the graphical user interface on the integrated display device,
(iv) receive a prompt input in a natural language format from a user to the input field, wherein the prompt input comprises a molecular target identification and an experiment objective,
(v) feed the prompt input to the trained neural network, wherein the trained neural network outputs the molecular experiment protocol having a highest probability of meeting the experiment objective,
(vi) generate material identifications using the prompt input and the assay function data,
(vii) render the molecular experiment protocol and the material identifications on the graphical user interface, wherein (A) the material identifications comprise an antibody and a fluorophore, and (B) the molecular experiment protocol comprises an imaging device identification and imaging device settings.

2. The system for cellular experiments of claim 1, wherein:
(a) the system further comprises an imaging device configured with the imaging device settings;
(b) a user loads the molecular target, the antibody, and the fluorophore into the imaging device; and
(c) the imaging device is activated to generate microscopy image data of the molecular target.

3. The system for cellular experiments of claim 1, wherein the imaging device identification is an imaging device that performs one or more of western blotting, widefield microscopy, structured illumination microscopy, widefield structured illumination microscopy, confocal laser scanning microscopy, super resolution structured illumination microscopy, or stochastic optical reconstruction microscopy.

4. The system for cellular experiments of claim 1, wherein:
(a) the antibody is a first primary antibody and the fluorophore is a first fluorophore;
(b) the material identifications further comprise a second primary antibody, a secondary antibody, and one or more additional fluorophores; and
(c) the molecular experiment protocol further comprises an order for reacting the first primary antibody with the first fluorophore and the second primary antibody or secondary antibody with the additional fluorophores.

5. The system for cellular experiments of claim 1, wherein the molecular experiment protocol further comprises instructions for cell culturing, cell fixation, cell blocking, antibody incubation, and cellular washing.

6. The system for cellular experiments of claim 1, wherein the molecular experiment protocol comprises a plurality of imaging device identifications and imaging device settings for each imaging device identification.

7. The system for cellular experiments of claim 1, wherein the molecular experiment protocol further comprises instructions for utilizing a selected buffer, a reagent, and mounting media.

8. The system for cellular experiments of claim 1, wherein the prompt input further comprises a hypothesis and executing the executable code further causes the processor to render a hypothesis refinement on the integrated display device.

9. The system for cellular experiments of claim 1, wherein:
(a) the prompt input comprises an inquiry for data charactering the molecular target identification and a molecular pathway; and
(b) executing the executable code further causes the processor to render on the integrated display device, a natural language description of the molecular target function in the molecular pathway.

10. The system for cellular experiments of claim 1, wherein the molecular target identification comprises one or more of Poly (ADP-ribose) polymerase 1 (PARP1), MRE11, RAD50, Nijmegen breakage syndrome 1 (NBS1), Replication Protein A (RPA), Bloom syndrome protein (BLM), Breast cancer 1 (BRCA1), CtBP Interacting Protein or RB Binding Protein 8 (CtIP), RAD51, RAD52, RAD54, FANCN, BRCA2, DSS1, FANCJ (BRIP1 or BACH1), ataxia-telangiectasia mutated (ATM), Tat-interactive protein, 60 kDa (TIP60), Checkpoint kinase 2 (CHK2), BRCA1-Associated RING Domain protein 1 (BARD1), HERC2, H2AX, MDC1, Histone protein 2A (H2A), RNF168, UBC13, Mms2, RNF8, P53-binding protein 1 (53BP1), deubiquitinase DUB (USP28), P53, GADD45, cyclin-dependent kinase inhibitor 1A (aCDKNIA), CIP1, Ccdc98, BRCC36, BRCC45, SUMO.Ub, Small Ubiquitin-like Modifier (SUMO), RNF4, MDC1, and UBC13.

11. A system for cellular experiments comprising:
(a) a design software module comprising a large language model and a trained neural network that is trained to generate material identifications and molecular experiment protocols, wherein the trained neural network is trained using protein-antibody compatibility data and antibody-fluorophore compatibility data;
(b) a computing device comprising a processor, an integrated display device, and a memory device storing executable code that, when executed, cause the first processor to:
(i) generate a graphical user interface, wherein the graphical user interface comprises an input field configured to receive natural language data,
(ii) render the graphical user interface on the integrated display device,
(iii) receive a prompt input, in a natural language format, entered by a user to the input field, wherein the prompt input comprises a molecular target identification and an experiment objective,
(iv) feed the prompt input to the large language model to generate molecular experiment design data,
(v) feed the molecular experiment design data to the trained neural network, wherein the trained neural network outputs a molecular experiment protocol and a plurality of material identifications,
(vi) render the molecular experiment protocol and the material identifications on the graphical user interface, wherein (A) the material identifications comprise an antibody compatible with the molecular target and a fluorophore compatible with the antibody, and (B) the molecular experiment protocol comprises an imaging device identification and imaging device settings.

12. A system for cellular experiments comprising a computing device that comprises a processor, an integrated display device, and a memory device storing a database of antibodies and fluorophores, reference assay function, fluorophore data comprising excitation and emission spectra, and executable code that, when executed, cause the processor to:
(a) generate a graphical user interface that comprises a pathway input field;
(b) render the graphical user interface on the integrated display device;
(c) receive a first input from an end user to the pathway input field, wherein the first input includes a molecular pathway;
(d) generate a pathway map for the molecular pathway;
(e) render the pathway map on the integrated display device, wherein the pathway map comprises (A) a plurality of active functional proteins that are connected within the pathway map, (B) callouts for reach active functional protein, wherein the callouts comprise a description of the one or more active functional proteins, and (C) an assay guide tool input function;
(f) render, when the assay guide tool input function is selected, an assay guide tool on the integrated display device, wherein the assay guide tool comprises primary antibody selection options for up to three active functional proteins, wherein the options are filtered using reference assay function data to identify compatible antibodies;
(g) render a dye selection guide on the integrated display device, wherein the dye selection guide comprises a list of fluorophores filtered using the reference assay function data and fluorophore data and based on user input of a selected wavelength;
(h) receive input by the user identifying one or more selected antibodies, one or more selected fluorophores, and an imaging device to be used for imaging;
(i) evaluate the compatibility of the selected fluorophore with the imaging device and other selected components using a trained neural network; and
(j) generate molecular experiment protocol comprising the selected one or more active functional proteins, the selected antibodies, the selected fluorophores, and an experiment protocol, wherein the experiment protocol comprises imaging device settings for the selected imaging device.

13. The system for cellular experiments of claim 12, wherein the molecular pathway is one or more of DNA damage and repair, cancer growth and spread, cell cycle, cell death, and mitochondria.

14. The system for cellular experiments of claim 12, wherein the molecular pathway is a non-homologous end joining double strand break pathway or a homologous recombination double strand break pathway.

15. The system for cellular experiments of claim 12, wherein the dye selection guide displays a list of available dyes at the user wavelength selection and complying with the reference assay function data.

16. The system for cellular experiments of claim 12, wherein the molecular experiment protocol further comprises a first secondary antibody selection, a primary antibody selection, a second secondary antibody selection, a second primary antibody selection, and a third secondary antibody selection.

17. The system for cellular experiments of claim 12, wherein the molecular experiment protocol further comprises a second fluorophore and a third fluorophore, wherein the second fluorophore is bound to the primary antibody selection and the third fluorophore is bound to the second or third secondary antibody selection.

18. The system for cellular experiments of claim 12, wherein:
(a) the system further comprises the selected imaging device configured with the imaging device settings;
(b) a user loads the molecular target, the antibody, and the fluorophore into the selected imaging device; and
(c) the selected imaging device is activated to generate microscopy image data of the selected one or more active functional proteins.

19. The system for cellular experiments of claim 12, wherein the imaging device is an imaging device that performs one or more of western blotting, widefield microscopy, structured illumination microscopy, widefield structured illumination microscopy, confocal laser scanning microscopy, super resolution structured illumination microscopy, or stochastic optical reconstruction microscopy.

20. The system for cellular experiments of claim 12, wherein the experiment protocol further comprises instructions for utilizing a selected buffer and a reagent.
